(19) United States
(12) Patent Application Publication
(10) Pub. No.: US 2021/0228300 A1
(43) Pub. Date: Jul. 29, 2021

(54) IMAGING DRAPES, PACKAGING FOR DRAPES, METHODS OF USE OF IMAGING DRAPES, AND METHODS FOR DEPLOYING DRAPE

(71) Applicant: MOLECULIGHT INC., Toronto (CA)

(72) Inventors: Ralph DaCOSTA, Toronto (CA); Garrett VERMEY, Toronto (CA); Nitesh MISTRY, Toronto (CA); Danielle DUNHAM, Toronto (CA); Simon TREADWELL, Toronto (CA); Sonia GULIA, Toronto (CA)

(73) Assignee: MOLECULIGHT INC., Toronto, ON (CA)

(21) Appl. No.: 17/053,607

(22) PCT Filed: May 9, 2019
(86) PCT No.: PCT/CA2019/000061

§ 371 Date: Nov. 6, 2020

Related U.S. Application Data
(60) Provisional application No. 62/669,009, filed on May 9, 2018.

(51) Int. Cl. A61B 46/10; A61B 6/00; A61B 6/10

(52) U.S. Cl. CPC A61B 46/10; A61B 6/107; A61B 6/485

(57) ABSTRACT

A drape for use with fluorescence-based imaging and white-light imaging includes a drape body. The drape body is configured to limit passage of electromagnetic radiation including light through the drape body to an interior imaging environment defined by the drape body such that electromagnetic radiation including ambient light within the interior imaging environment does not exceed a predetermined threshold. The drape also includes a connecting element permanently coupled to the drape body. The connecting element defines a hole in the drape body. The connecting element is configured to attach the drape to a portable, handheld imaging device.

### CROSS-REFERENCE TO RELATED APPLICATIONS

This patent application claims the benefit of priority to U.S. Provisional Application No. 62/669,009, filed on May 9, 2018, the entire content of which is incorporated by reference herein.

### TECHNICAL FIELD

The present disclosure relates to a drape for providing a portable imaging environment in which ambient light is reduced or limited for fluorescent imaging of a target within the portable imaging environment. The drape may be used with a portable, handheld fluorescent-based imaging device. The drape may also be used with white-light imaging and/or measuring devices and processes.

### INTRODUCTION

The section headings used herein are for organizational purposes only and are not to be construed as limiting the subject matter described in any way.

Recent advances in technology allow clinicians to quickly, safely, and easily visualize bacteria (and other biological components) in wounds and measure wounds at the point of care. This permits maximum insight for accurate treatment selection and accelerated healing in real time. Fluorescence-based imaging allows clinicians to focus on potentially harmful levels of bacteria. The technology is based on the detection and analysis of intrinsic fluorescence signals emitted by tissues and microbes (or by exogenous agents) when illuminated with specific wavelengths of light. Room lighting can interfere with the fluorescence signals emitted by the bacteria in wounds or on other surfaces that clinicians or others are imaging.

Fluorescence imaging can be enhanced and optimized when the imaging is performed in a darkened environment or in an environment with reduced or limited ambient light. This may be difficult to achieve when ambient light, such as sunlight, room lighting or other non-visible electromagnetic radiation (optical) interference is present in the imaging environment. Even when it may be possible to change ambient lighting, such as by turning off room lights or pulling shades, doing so may create other problems for clinicians, such as difficulty seeing. Further, one of the benefits of a portable, handheld imaging device is that it may be used at point-of-care, such as in the field treating armed forces, refugees, or others in need of care in outdoor conditions. In these scenarios, it is not possible to dim lights or pull shades. Thus, it would be desirable to create a portable imaging environment which can provide an environment suitable for imaging regardless of location.

### SUMMARY

The present disclosure may solve one or more of the above-mentioned problems and/or may demonstrate one or more of the above-mentioned desirable features. Other features and/or advantages may become apparent from the description that follows.

In accordance with various exemplary embodiments of the present disclosure, a drape is provided. The drape comprises a drape body, wherein the drape body is configured to limit passage of electromagnetic radiation through the drape body to an interior imaging environment defined by the drape body such that electromagnetic radiation within the interior imaging environment does not exceed a predetermined threshold. A connecting element is permanently coupled to the drape body and defines an opening in the drape body. The connecting element is configured to attach the drape to an imaging device.

In accordance with another aspect of the present disclosure, a system for fluorescence-based imaging of a target is provided. The system comprises a drape having a drape body configured to limit passage of electromagnetic radiation through the drape body to an interior imaging environment defined by the drape body such that electromagnetic radiation within the interior imaging environment does not exceed a predetermined threshold. A connecting element is permanently coupled to the drape body and defines an opening in the drape body. The connecting element is configured to attach the drape to an imaging device. The system also includes a portable, handheld imaging device, wherein the imaging device includes a portion with a lens and at least one violet light source, and wherein the connecting element is configured to encompass an area surrounding the lens and the at least one light source to position the light source within the interior space of the drape body.

The portable, handheld imaging device may also include a white light source and may be configured for white light imaging.

The imaging target may be a human or animal body part and may include a wound, lesion, cut, incision, tumor, or other abnormality on or in the body part.

In accordance with a further aspect of the present disclosure, a drape comprises a drape body configured to move between a closed configuration and an open configuration. The drape body is substantially planar in the closed configuration and in the open configuration, the drape body has a shape that is substantially cylindrical, substantially rectangular, a truncated cone, or a truncated pyramid. A connecting element is coupled to the drape body and is configured to attach the drape to a portable, handheld imaging device. At least one valve is configured to pass air into the drape as the drape moves from the closed configuration to the open configuration.

In accordance with yet another aspect of the present disclosure, a method of creating a portable imaging environment is disclosed. The method comprises positioning a portable, handheld imaging device relative to an opening of a darkening drape while the drape is in a collapsed condition and connecting a frame of the drape to the imaging device. The method also includes moving the imaging device and connected drape away from a packaging element supporting the drape in the collapsed condition.

Another aspect of the present disclosure provides a method of obtaining a fluorescence-based image or video without changing ambient lighting conditions. The method comprises removing a cover from an opening of a darkening drape, attaching a portable, handheld imaging device to the drape while the drape is connected to a packaging element, and moving the imaging device and attached drape away from the packaging element to deploy the drape. The method also includes positioning an open, bottom end of the drape over an area of a body to be imaged, contouring a portion of the drape around the body area to be imaged to create a darkened imaging environment around the body area to be imaged, wherein the drape is configured to limit passage of ambient light through the drape into the darkened imaging environment, and capturing a fluorescent image or a fluorescent video of the body area with the imaging device attached to the drape.

Yet another aspect of the present disclosure provides a dispensing element configured to store a darkening drape. The dispensing element comprises a substantially planar base portion, and at least one restraining element configured to engage a shaping element of a darkening drape.

A further aspect of the present disclosure provides a darkening drape system. The system comprises a darkening drape having a drape body, wherein the drape body is configured to limit passage of ambient light through the drape body to an interior imaging environment defined by the drape body. The darkening drape also includes a frame configured to attach the drape to an imaging device, and at least one shaping element configured to maintain the drape in a position outside a field of view of the imaging device during imaging. The system also comprises a drape dispensing element. The drape dispensing element includes a substantially planar base portion, and at least one restraining element configured to engage the at least one shaping element of the darkening drape.

In accordance with yet another aspect of the present disclosure, a method of obtaining a measurement of a target without changing ambient lighting conditions is provided. The method comprises removing a cover from an opening of a darkening drape, attaching an imaging device to the drape while the drape is connected to a packaging element, and moving the imaging device and attached drape away from the packaging element to deploy the drape. The method also comprises positioning an open, bottom end of the drape over an area of a body containing the target to be measured, contouring a portion of the drape around the body area containing the target to be imaged to create a darkened environment around the body area containing the target to be measured, wherein the drape is configured to limit passage of electromagnetic radiation through the drape into the darkened environment, and measuring the target on the body area with a portable, handheld imaging device attached to the drape.

Additional objects and advantages will be set forth in part in the description which follows, and in part will be obvious from the description, or may be learned by practice of the present disclosure. The objects and advantages may be realized and attained by means of the elements and combinations particularly pointed out in the appended claims and their equivalents.

It is to be understood that both the foregoing general description and the following detailed description are exemplary and explanatory only and are not restrictive of the present disclosure and claims.

### BRIEF DESCRIPTION OF THE DRAWINGS

The present disclosure can be understood from the following detailed description either alone or together with the accompanying drawings. The drawings are included to provide a further understanding and are incorporated in and constitute a part of this specification. The drawings illustrate one or more exemplary embodiments of the present disclosure and together with the description serve to explain various principles and operations.

FIG. 1 is a perspective view of an example imaging device, capable of fluorescence-based and/or white-light imaging, and to which a drape, in accordance with the present disclosure, can be attached or connected.

FIGS. 2 and 3 are a perspective view and a sectional view, respectively, of a first example embodiment of a drape in a deployed configuration or expanded condition, in accordance with the teachings of the present disclosure.

FIG. 4 is a partial view of the drape of FIG. 2, showing a shaping element embedded in the drape in accordance with the teachings of the present disclosure.

FIGS. 5A-5C show a second example embodiment of a drape in a closed, undeployed, condition (FIGS. 5A-5B) and in an open, deployed condition (FIG. 5C), in accordance with the present disclosure.

FIGS. 6A-6C show a third example embodiment of a drape in accordance with the present disclosure, with FIG. 6A showing the drape in use with an imaging device, FIG. 6B showing the drape with the imaging device removed, and FIG. 6C showing the drape in a closed, undeployed condition.

FIG. 7 is a perspective view of a fourth example embodiment of a drape, in use with an imaging device, in accordance with the present disclosure.

FIGS. 8A-8I show various views of an example embodiment of a connecting element of a drape in accordance with the present disclosure.

FIGS. 9A-9C show a drape connected to a portable, handheld imaging device in an undeployed and deployed configuration, respectively, in accordance with the present disclosure.

FIGS. 10A-10E show a dispensing/packaging element with and without the drape of the present disclosure.

FIGS. 11A-11C show an exemplary cover for the opening in the connecting element of the drape, in accordance with the present disclosure.

FIGS. 12A-12F show a fifth example embodiment of a drape unconnected to (FIGS. 12A-120) and connected to (FIGS. 12D-12E) a portable, handheld imaging device in accordance with the present disclosure.

FIGS. 12G-12H show the example portable, handheld imaging device connected to the drape of FIGS. 12D-12F in accordance with the present disclosure.

FIG. 12I shows another example imaging device that may be used with an imaging drape in accordance with the present disclosure.

FIGS. 13A-13C show an example portable, handheld imaging device to be used with a drape, such as the drapes of FIGS. 2-7, in accordance with the present disclosure.

FIG. 14 is another example of a portable, handheld imaging device to be used with a drape in accordance with the present disclosure.

FIGS. 15A-15B illustrate a surgical shield or sterile sheath disposed over the portable, handheld imaging device of FIG. 14 in accordance with the present disclosure.

FIG. 15C illustrates the sterile shield of FIGS. 15A-15B coupled to a connecting element of a drape in accordance with the present disclosure,

FIGS. 16A-16D illustrate various features that couple the sterile shield for the portable, handheld imaging device of FIGS. 15A-B to the drape of FIG. 15C in accordance with the present disclosure.

### DESCRIPTION OF VARIOUS EXAMPLE EMBODIMENTS

This description and the accompanying drawings illustrate example embodiments of a drape, imaging devices connectable to various drapes, and structures for connecting drapes to surgical sheaths and are example only and should not be taken as limiting. Various mechanical, compositional, structural, electrical, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

When performing fluorescence imaging, it is desirable to have a dark or darkened environment (i.e., an environment with limited ambient light) to optimize the fluorescent (FL) image obtained. This may be difficult to achieve when sunlight, room lighting or other non-visible electromagnetic radiation (optical) interference is present in the imaging environment. The present disclosure provides an imaging drape that can be positioned over a target to be imaged to reduce or limit ambient light around the target. In some embodiments, the imaging drape can conform around an object to be imaged, including any part of the human body or animal body. The drape reduces or limits an amount of ambient light and other electromagnetic radiation that is within an imaging environment defined by an interior of the drape. Limiting the amount of electromagnetic radiation, including the amount of ambient light, that passes through the drape into an environment contained within the drape creates a darkened environment within the drape for performing imaging. While beneficial for fluorescent-based imaging, the darkened environment created by the drape is also useful in white-light imaging (to reduce or prevent glare) and in taking measurements (digital, for example, using an image of the target, such as a wound, to obtain measurements). While drapes in accordance with the present disclosure are generally discussed herein for providing a darkened imaging environment, it is within the scope of the present disclosure for a drape to be configured to provide a portable imaging environment that reduce or limits glare or provides an anti-reflective effect rather than providing a darkened environment. Such a drape may be useful for white light imaging or measuring of imaging targets. Similarly, drapes in accordance with the present disclosure may both provide a darkened imaging environment and reduce glare. Drapes in accordance with the present disclosure may provide a darkened imaging environment and an anti-reflective effect. It is also within the scope of the disclosure that the drape may provide a reduced ambient light environment within an interior of the drape. For example, the drape may provide a frosted effect so that some, but not all, of the light penetrates through the drape and into the interior of the drape. In some examples, the drape is coated with a fluoropolymer coating to provide the anti-reflective effect. The fluoropolymer coating may be a single-layer interference coating.

In accordance with one aspect of the present disclosure, a drape configured to reduce or limit ambient light (i.e., a darkening drape) is lightweight, compact, and portable. The portable drape is easy for personnel, such as medical or emergency personnel or other health care workers, to carry a supply of drapes with them for use with a portable, handheld imaging device. In this way, a darkened imaging environment can be created under any conditions, indoors or outdoors, without concern about ambient lighting or other electromagnetic radiation interfering with the process of obtaining an image of a target, such as for example, a fluorescent image of a wound or injury. Although the imaging drape may move between a collapsed/closed/undeployed condition and an expanded/opened/deployed configuration, it is also contemplated that the drape may not be collapsible and is packaged in a ready-to-use configuration.

The drape may also be used in a more controlled setting, such as a doctor's office or surgical suite, where darkened imaging conditions are desirable but reducing overall ambient lighting is not desirable. Use of the drape creates a portable, darkened imaging environment that allows fluorescence imaging to be performed in any lighting condition resulting in good fluorescence image (or video) quality without background light interference. As mentioned above, the drape in accordance with the present disclosure allows the use of a portable, handheld imaging device to be used at the point of care without needing to turn off the lights Such a portable, handheld imaging device may include one or more of fluorescence-based imaging capability, white-light imaging capability, and target measuring capability. Examples of such imaging devices are described in U.S. Pat. No. 9,042,967, filed May 20, 2009, issued May 26, 2015, and entitled “Device and Method for Wound Imaging and Monitoring.” The entire content of U.S. Pat. No. 9,042,967 is incorporated herein by reference. This device may be used in conjunction with the darkening drape disclosed herein, in both clinical and non-clinical applications, when obtaining images of a target area using a handheld imaging device is useful. Additional examples of imaging devices that may be used with a darkening drape in accordance with the present disclosure are disclosed in U.S. Provisional Patent Application No. 62/793,842 (1142.0010-00999), filed on Jan. 17, 2019 and entitled “Modular System for Multi-Modal Imaging and Analysis”; U.S. Provisional Patent Application No. 62/793,846 (1142.0011-00999), filed on Jan. 17, 2019 and entitled “Endoscopic System for Visualization of Disease”; and U.S. Provisional Patent Application No. 62/793,764 (1142.0012-00999), filed on Jan. 17, 2019 and entitled “Devices, Systems, and Methods for Tumor Visualization and Removal”, the entire content of each of which is incorporated herein by reference.

As noted above, the drape can be used for fluorescent-based imaging, white-light imaging, and/or measuring. These processes may be done sequentially or at separate times. In addition, the user can perform specific tasks using the handheld FL imaging device such as, for example, image-guided sampling and/or cleaning and debridement of a wound, by raising a side of the drape for the intervention during FL imaging.

In accordance with the present teachings, the darkening drape is a one-time use disposable accessory that can be attached to a portable, handheld imaging device to provide users with a portable, darkened imaging environment to achieve the necessary lighting conditions for performing fluorescence imaging and/or white light imaging. This will allow imaging of any target that can be covered by, at least partially encompassed by, and/or partially surrounded by the interior of the drape. For example, in clinical applications, the drape may be manipulated to encompass an imaging site anywhere on the human body. The drape may be manipulated to change size or shape to encompass, for example, a limb on a patient that contains an imaging target such as a wound. In another example, the imaging drape and imaging device may be used to image excised tissue specimens, such as tumors, cancerous tissue, or other abnormal tissue. This may be done, for example, in a pathology lab immediately after removal of the specimen from the patient. In such a case, the drape may be sized and/or shaped to completely cover or otherwise encompass the excised tissue specimen during imaging with the handheld imaging device.

The drape may be provided and packaged as a sterile unit and one example method of deployment maintains the interior side (patient side/patient contacting side) of the drape in a sterile condition. In some embodiments, a sterile sheath/surgical drape/sterile shield is disposed around part or all of the imaging device in order to maintain the sterile environment within the interior of the drape and/or keep the imaging device from being contaminated. The terms sterile sheath, surgical sheath, surgical drape, and sterile shield are used interchangeably herein to indicate a sheath or drape that is used for sterility purposes/to reduce contamination by encompassing a part or all of the imaging device.

A drape, such as a darkening drape or an imaging drape, as used herein, refers to a drape that is configured to reduce, limit, and/or alter an amount and/or characteristic of ambient light that passes through the drape to an interior of the drape, wherein the interior of such a drape may define a portable imaging environment. Thus, a drape that limits or reduces the amount of ambient light passing through the drape body may provide a darkened imaging environment. Alternatively, a drape that alters a characteristic of ambient light may provide an imaging environment with a quality associated with the change, for example an imaging environment that provides less glare or an anti-reflective imaging environment. The term imaging drape encompasses a darkening drape as well as any other type of drape used to create an altered or tailored imaging environment.

In cases where a surgical drape is used to cover part or all of an imaging device, a darkening drape or imaging drape may be attached to the surgical drape, for example, by a connecting element on one or both of the darkening drape (or imaging drape) and the surgical drape. Additionally or alternatively, the inventors contemplate that a surgical drape and imaging drape may be formed as a single drape, for example, where a portion of the drape is suitably shaped and/or made of a material to reduce or limit ambient light.

In some embodiments, the drape may include a retaining element that must be broken to remove the drape from the imaging device. Once the retaining element is broken, it is no longer configured to permit attachment of the drape to the imaging device. Although the drape is intended as a disposable device, to be discarded after a single use, it is within the scope of this application that the drape may be used more than once.

In such a case, it is possible that the drape is made from a material that is easily sterilizable. However, the inventors understand that users may, for whatever reasons, re-use a non-sterilizable drape or re-use a drape that is capable of being sterilized without doing so.

In accordance with the present teachings, the drape is made of a material that reduces or limits an amount of electromagnetic (EM) radiation that passes through the drape, from an exterior of the drape to an interior imaging environment defined by the drape. The amount of EM radiation that is blocked, or prevented from passing through the drape, is sufficient to prevent interference with imaging modes of the portable, handheld device, such as a fluorescent imaging mode and a white light imaging mode. The drape material substantially reduces the amount of ambient light transmitted through the drape to an interior of the drape that defines the imaging environment such that the imaging device functions optimally. Functioning optimally may include, for example, the use of an imaging device in white light and/or fluorescence imaging modes, without an ambient light sensor of the portable, handheld imaging device being triggered to indicate, for example, that too much ambient light is present. Alternatively, it may include, for example, the use of an imaging device in white light and/or fluorescence imaging modes, wherein an ambient light sensor is triggered to indicate that lighting conditions are sufficient to permit imaging without interference from ambient light. In each case, the imaging device is able to produce a high quality image. The drape may block substantially all visible light (i.e., 400 nm-700 nm wavelengths of light) from entering the interior of the drape. The inventors have found that, to provide acceptable quality fluorescent images, the amount of ambient (visible) light in the imaging environment should be less than about 402 mW/m², In some embodiments, the drape may be configured to provide an imaging environment having less than about 402 mW/m² of ambient light. In other embodiments, the drape may be configured to provide a portable imaging environment having fess than about 401.1 mW/m², less than about 350 mW/m², less than about 300 mW/m², less than about 250 mW/m², less than about 200 mW/m², fess than about 190 mW/m², and/or less than about 185 mW/m² of ambient light. In one example embodiment, fluorescent imaging was performed in a well-lit hospital room. The amount of ambient light in the hospital room was measured to be 290,200 mW/m². The amount of ambient light measured in the portable darkened imaging environment formed by a drape in accordance with the present disclosure was 184.6 mW/m², Thus, in this example, the imaging drape prevented about 99.94% of ambient light in the external imaging environment from entering the portable imaging environment defined by the interior of the imaging drape.

As discussed further below, suitable materials that may be used for the drape include plastics and plastic composites, polymers, rubber materials, paper materials, vinyl materials, and cloth materials. In addition, the drape material may be coated or impregnated with one or more materials configured to reduce or substantially block transmission of EM radiation, including ambient or visible light. In other embodiments, the drape may block substantially all visible light or EM radiation that may be interfering with the fluorescence imaging being performed. The drape may be made from a single material or multiple materials. The drape may be formed in multiple portions or may be a single piece made by, for example, injection molding.

The imaging device may have an ambient light sensor. The ambient light sensor may sense the level of ambient light in the environment in which the image will be taken. If the amount of ambient light present in the imaging environment is too high or too great, the sensor may flash a warning, turn amber or red in color, or otherwise provide a warning/user feedback that conditions are not appropriate for fluorescence imaging. If the amount of ambient light present in the imaging environment is acceptable for fluorescence imaging, the sensor may flash green for “ready” or “good” or give another indication or feedback indicative that conditions are adequate for fluorescence imaging. It is within the scope of the present application that the ambient light sensor may provide other warnings or otherwise sense different gradations or amounts of ambient light present in the imaging environment. For example, the ambient light sensor may have different ambient light settings/acceptable thresholds for different types of imaging when sensing an amount of ambient light that is acceptable to provide a clear image. For example, it may be desirable to have less ambient light when taking a fluorescent image than when taking a white-light image. In accordance with one aspect of the disclosure, for fluorescent imaging, the ambient sensor may be set to indicate that imaging conditions are acceptable when ambient light present is less than 402 mW/m². Alternatively, the ambient sensor may warn that fluorescent imaging conditions are not acceptable when ambient light in the imaging environment is more than about 402 mW/m² of ambient light.

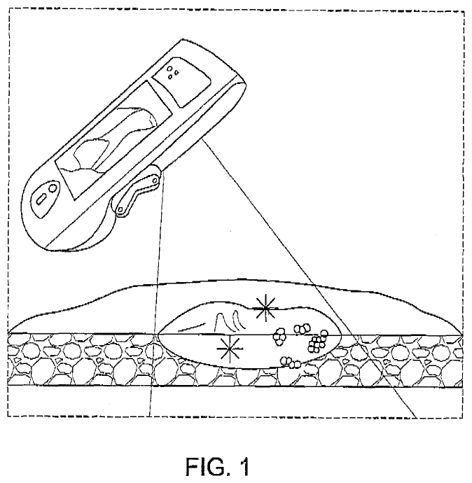

In accordance with one aspect of the present disclosure, the drape may be used with a portable, handheld imaging device, such as the exemplary device shown in FIG. 1. Examples of such imaging devices are described in U.S. Pat. No. 9,042,967, filed May 20, 2009, issued May 26, 2015, and entitled “Device and Method for Wound Imaging and Monitoring.” The entire content of U.S. Pat. No. 9,042,967 is incorporated herein by reference. The patent describes the characteristics of imaging devices to be used with the drape disclosed herein, as well as methods of imaging, both white light and fluorescence-based imaging, that may benefit from the drape of the present disclosure. Additional examples of imaging devices that may be used with a darkening drape in accordance with the present disclosure are disclosed in U.S. Provisional Patent Application No. 62/793,842 (1142.0010-00999), filed on Jan. 17, 2019 and entitled “Modular System for Multi-Modal Imaging and Analysis”; U.S. Provisional Patent Application No. 62/793,846 (1142.0011-00999), filed on Jan. 17, 2019 and entitled “Endoscopic System for Visualization of Disease”; and U.S. Provisional Patent Application No. 62/793,764 (1142.0012-00999), filed on Jan. 17, 2019 and entitled “Devices, Systems, and Methods for Tumor Visualization and Removal”, the entire content of each of which is incorporated herein by reference.

In accordance with one aspect of the present disclosure, the drape may have a drape body formed of a material or materials configured to reduce, limit, block, or substantially prevent the passage of EM radiation, such as ambient or visible light. The drape is configured to create an imaging environment for any imaging target which can be partially or fully surrounded by the drape. For example, in various example embodiments, the drape is configured to conform to the shape of any part of a patient's body, thus allowing imaging of that part of the body. For example, the drape may be shaped to encompass a foot, a hand, an arm, etc., and create an imaging environment around that body part. If the drape cannot fully encompass a target, it may be attached or adhered to an area surrounding the target for forming an enclosed imaging environment. A portion of the drape body, such as a central portion, may include a mounting device, such as a connecting element or frame that is configured to allow the drape to be mounted onto the imaging device or otherwise connected to the imaging device. This connection component may be referred to as a connecting element, frame portion, or a mounting element. For example, the connecting element may include a frame portion configured to receive a portion of the handheld, portable imaging device, such as a lens portion (also referred to herein as an imaging head or optical head) which includes a lens, one or more excitation light sources (for example, LEDs) to illuminate the target for fluorescence imaging (the excitation light sources may include, for example, ultraviolet light, violet/blue light, other wavelengths of visible light, near infrared light, and/or infrared light and may be provided for fluorescence imaging), and a sensor for sensing ambient light conditions. Depending on the configuration of the imaging device, other components, such as a white light source for illumination for white light imaging, an image sensor, and/or a spectral filter may be included in the portion of the imaging device that is received in the frame portion of the drape.

The connecting element of the drape defines an opening in the drape, and when the drape is in a deployed or expanded configuration, the opening provides access into an interior of the drape. The interior of the deployed drape forms a portable imaging environment that is darkened (e.g., has less ambient light) relative to ambient environment outside of the drape. A portion of the imaging device (e.g., lens portion, imaging head, or optical head) may be received in a press-fit manner in the connecting element defining the opening, such that ambient light does not pass through the opening into the portable imaging environment. This press-fit relationship may be defined as a sealing relationship as it seals out ambient light. Other means may be used to sealingly connect the portion of the imaging device to the connecting element such as for example, hook and loop fasteners, sealants, adhesives, clips, and other mechanical and/or chemical fastening elements.

In some example embodiments, the drape may include shaping elements to facilitate conforming a shape of the drape to the target to be imaged such that the area (target) to be imaged is substantially within the imaging environment (e.g., the interior of the drape) or substantially surrounded by the interior of the drape. The shaping elements may be placed around a perimeter of the drape (e.g., bands), may run lengthwise through the drape (e.g., extend from a top portion of the drape to a bottom or lower portion of the drape), may extend diagonally across the drape body, or may be a combination of such shaping elements. The shaping elements may comprise, for example, wire, drawstrings, or other elements that are deformable or otherwise can facilitate changing the shape and/or size of the drape.

In some embodiments, the shaping elements may also serve to maintain a shape of the drape to provide a clear field of view or clear imaging field within the imaging environment (e.g., within the body of the drape when in the deployed or expanded configuration). That is, the shaping elements may be sized and positioned to maintain the drape body in a position which does not impede or otherwise affect the field of view (FOV) of the lens portion (imaging head or optical head) of the imaging device when the imaging device is mounted to or connected to the drape via the connecting element. In this way, the shaping elements hold the drape body out of the field of view of the lens, such that the drape body does not extend into the field of view between the lens and the target to be imaged.

In some example embodiments, a bottom portion of the drape may be provided with a mechanism to create light-reducing contact between an area surrounding the target. For example, the bottom of the drape may be provided with weighted elements to hold the bottom of the drape in contact with a surface. Alternatively, the bottom of the drape could be provided with one or more fixing elements or mechanisms to provide a connection between a surface around the target and the drape. The surface around the target may be the actual surface which contains the target or a secondary surface, such as a supporting surface or a surface added for sterility, such as a surgical drape. The fixing element(s) or mechanism(s) may include, for example, magnets, snaps, hook and loop closures, adhesive, and other suitable fastening mechanisms. Additionally or alternatively, the bottom of the drape may be provided with a mechanism to tighten the drape around the imaging target. For example, the drape may include a drawstring, hook and loop closure, snaps, or other mechanism to reduce a diameter/perimeter of the bottom of the drape and to draw it close around the imaging target.

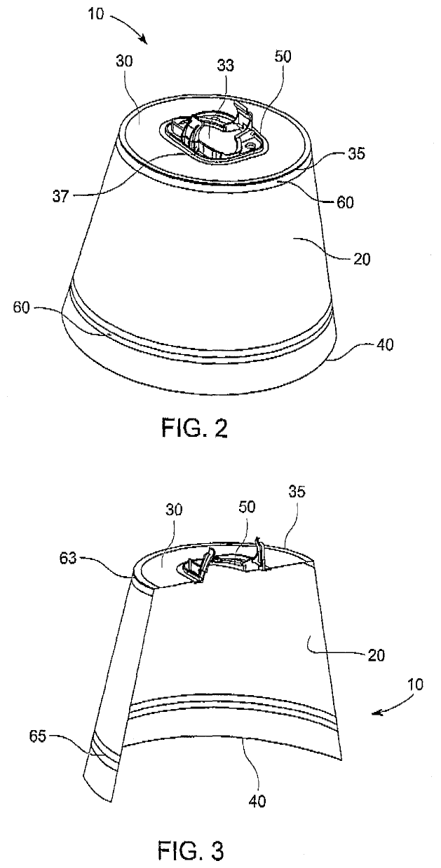

Turning now to the drawings, FIG. 1 shows an example of a portable, handheld imaging device imaging a wound in tissue without the use of an imaging drape. In accordance with one aspect of the present disclosure and as shown in the example embodiment of FIGS. 2-4, an imaging drape 10 includes a body 20 with a top portion 30 that is defined by a top edge 35, as shown in FIGS. 2 and 3, Top portion 30 may include an opening 33 that is within or defined by a connecting element 50 which is positioned in top portion 30 of the drape 10. As discussed further below, connecting element 50 may receive and secure the imaging device to the drape. In various example embodiments, the connecting element is permanently attached to the top of the drape during manufacturing such that the drape is not intended to be removed from the connecting element. Although not illustrated herein, it is within the scope of the present disclosure that the drape and a type of connector element may be provided separately, so that the connector element can be reused with disposable drapes. In such an embodiment, it would be necessary to provide a means for attaching the drape to the connector, such as for example, elastic, adhesive, etc.

As discussed further below, drape 10 moves from an undeployed configuration, to an open, deployed configuration. In the open, deployed configuration, body 20 extends radially outward from top edge 35 to a bottom edge 40, as shown in FIG. 2. In some embodiments, such as shown in FIGS. 2-4, a diameter of bottom edge 40 is greater than a diameter of top edge 35, when measured in the deployed configuration. However, it is contemplated in some embodiments that the diameter of the bottom edge 40 may be substantially the same as the diameter of the top edge 35, or may even be manipulated, during deployment, to have a diameter smaller than the top edge 35. Further, while the measurements discussed above are provided in terms of radial measurements (e.g., diameter, etc.), it is contemplated that the top edge and bottom edge of the drape may not always form circles, but instead may form triangles, squares, rectangles, ovals, etc. In such cases, the bottom edge of the drape may define a larger area than an area defined by (e.g., enclosed by) the top edge of the drape, an area of the same size as that defined by the top edge of the drape, or an area of less size (after deployment and manipulation). Although disclosed herein as being movable from a planar, undeployed condition to an expanded, deployed condition, it is within the scope of the present disclosure that the imaging drape is not collapsible, foldable, or otherwise changeable for storage and is provided in a ready-to-use configuration.

A geometry of the drape is configured so that the sides of the drape body do not fall into the field of view of the imaging device. In some embodiments, a diameter of top edge is about 60-80%, and in some cases about 70% of a diameter of bottom edge 40, to help prevent the sides of the drape from collapsing inward in the deployed configuration. In some embodiments, the diameter of top edge 35 is about 260 mm or larger, the diameter of bottom edge 40 is about 370 mm or larger, and a height of the drape in the deployed configuration (from top edge 35 to bottom edge 40) is about 260 mm to about 300 mm and in some cases about 280 mm. A longer drape would require larger diameters for the top and bottom edges so that the drape does not collapse into the field of view of the imaging device. The diameter of bottom edge 40 may range from about 381 mm to about 458 mm. Body 20 may extend radially outward from top edge 35 due to one or more shaping elements, as discussed further below.

When in the deployed configuration, top portion 30 extends outward from connecting element 50 in a substantially radial direction, and the remainder of body 20 forms a skirt that extends substantially vertically from top edge 35 towards bottom edge 40. The skirt portion of body 20 that extends between top edge 35 and bottom edge 40 may be flexible or rigid.

In the embodiment of FIGS. 2 and 3, body 20 has a truncated cone shape in the open, deployed configuration. However, it is also contemplated that body 20 may assume a variety of shapes including, for example, a cylinder, an umbrella, or a rectangle when in the open, deployed configuration. It is also contemplated that body 20 may be a truncated pyramid with, for example, a rectangle, square, pentagon, or hexagon base. In some embodiments, body 20 may be a truncated cone with either a circular or elliptical base. Body 20 may be formed of a lightweight and flexible material. In some embodiments, body 20 is formed of a thin plastic film such as, for example, polyethylene or polyolefin. The plastic film may range from about 0.05 mm to about 2 mm thick. The thin plastic film material of the drape allows it to be light weight, to be quiet, to contour to the patient's body, and to be easily packaged, Additionally or alternatively, body 20 may be formed of a paper material, a vinyl material, or a cloth material. However, it is also contemplated that body 20 may be formed of any other suitable and/or well-known materials or combinations of materials.

The material of drape body 20 may be a dark color, such as black, to prevent light from entering, thus providing a darkened imaging environment for FL imaging. The interior of the drape may have a matte and/or non-reflective finish to reduce any glare or reflection from the excitation light with which the target is illuminated during imaging. Additionally, the interior and/or exterior of the drape may include a coating that is configured to limit the passage of electromagnetic radiation, such as infrared light or ultraviolet light. Alternatively, the interior and/or exterior of the drape may include a coating that is configured to limit passage of some, but not all, visible light. For example, the coating may provide a frosted effect on the drape. The interior of the drape may also be sterilized during manufacturing or prior to use.

In the embodiment of FIGS. 2 and 3, top edge 35 is a weld between the top 30 and a skirt portion that forms body 20 and forms a raised edge or weld on body 20. One or more shaping elements 60, for example wires, may be used to form the edge of top edge 35, However, it is also contemplated that body 20 may not include such a discrete edge. Instead, for example, body 20 may continuously extend radially outward and downward from connecting element 50 toward bottom edge 40 (for example, a type of umbrella shape with the connecting element positioned at the apex of the umbrella shape).

Top portion 30 of drape 10 may have a substantially planar shape and may extend in a substantially horizontal manner away from connecting element 50 when drape 10 is deployed. In this way, the field of view of the lens portion (imaging head or optical head) of the imaging device, connected to the frame and “viewing’ the interior of the drape (i.e., the imaging environment), is kept clear, i.e., is not blocked by the body 20 of the drape 10. Such a substantially flat top portion of the drape is shown in the embodiment of the drape illustrated in FIGS. 2 and 3, Top portion 30 may be rigid or flexible.

As discussed above, drape 10 may include one or more shaping elements 60 that hold or support the drape material away from a central portion of the imaging environment created by the drape body. This allows a field of view of the imaging device, connected to the drape, to remain unobstructed by the drape body during imaging. These shaping elements may include, for example, wires, stiffeners, and other materials. In some embodiments, the shaping elements 60 may be formed of, for example, steel, aluminum, titanium, or plastic. In one example, shaping elements 60 are formed of 18-gauge mild steel wire hoops. It is also contemplated that shaping elements 60 are formed by seam in the material of drape body 20. In some embodiments, the material of drape body 20 is welded together to form the seam. In one example embodiment, the weld between top portion 30 and the skirt that forms body 20 may be sufficient to hold the body 20 out of the field of view (FOV) of the imaging device. In order to achieve this, the size of a diameter of the bottom portion of the drape must be larger than the top portion of the drape to prevent the sides of the drapes from collapsing inwardly on themselves. For example, the top diameter of the drape may be about 70% of the bottom diameter of the drape.

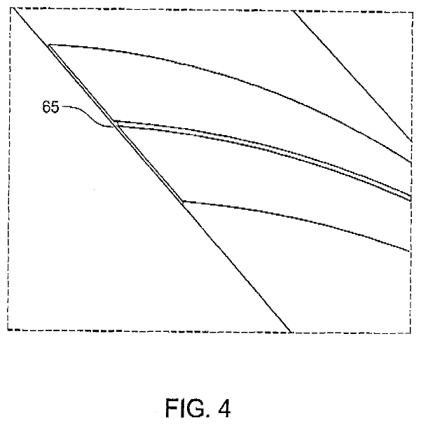

The shaping elements are also flexible to allow a shape of the drape body to be changed and capable of holding a changed shape to hold the drape body in a shape that, for example, has been contoured to a part of a patient's body to be imaged, such as a limb containing a wound or other target for imaging. In one example embodiment, shaping elements 60 may be disposed at the top of the drape and about 2.5″ from the bottom of the drape. For example, as shown in FIGS. 3 and 4, a first shaping element 63 is disposed at top edge 35 and a second shaping element 65 is disposed adjacent bottom edge 40. As shown, these shaping elements 63, 65 extend around a perimeter of the drape at each location. It is also contemplated that second shaping element 65 may be disposed along a bottom edge perimeter of drape 10, for example at bottom edge 40. First and second shaping elements 63, 65 are spaced a sufficient distance so that drape 10 will assume its desired shape in the open, deployed configuration.

Shaping elements 63, 65 are configured to be bent and manipulated so that drape body 20 will conform around the contours of an imaging target, such as a portion of a patient's body while maintaining the deployed shape of drape body 20 in order to create the ideal light conditions for FL imaging within the portable imaging environment.

For example, shaping elements 63, 65 may be wires that an operator can easily bend into a variety of shapes. Such may allow the drape to be bent so that it better conforms to a shape of the patient's body that is to be imaged. Additionally or alternatively, shaping elements 60 may help maintain a clear field of view for the imaging device by holding the drape body away from a center of the portable imaging environment created within the drape body.

FIG. 3 shows an embodiment of shaping elements 63, 65 having a circular shape. However, the shaping elements may comprise a variety of shapes, depending on the desired shape of drape body 20. Shaping elements 63, 65 may be disposed on an interior side or exterior side of drape body 20. In some embodiments, shaping elements 63, 65 may be embedded into the material of drape body 20. Although two shaping elements are depicted in the embodiment of FIGS. 2-4, it is contemplated that only a single shaping element may be used, or that more than two shaping elements may be used, for example, three, four, five, six or more shaping elements may be used.

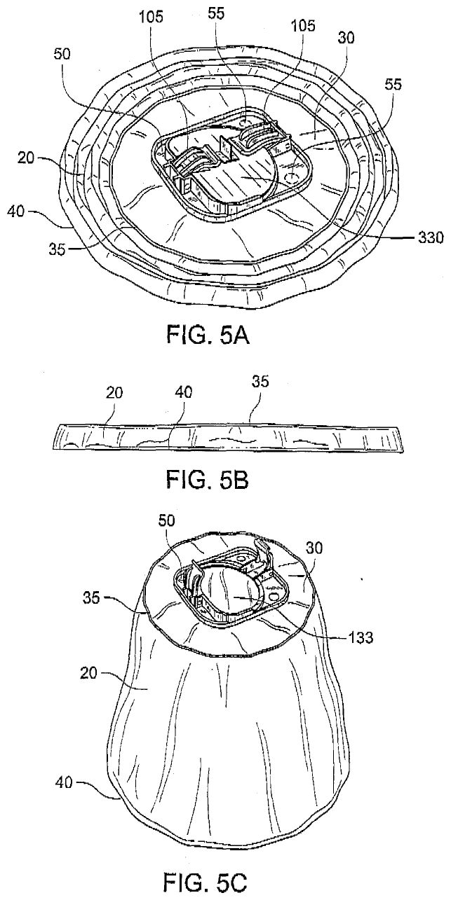

As shown in the example embodiment of FIGS. 2-4, the skirt or body portion 20 of drape 10 may be smooth and taut. Alternatively, as showing in FIGS. 5A-5C, the skirt or body portion 20 of the drape 10 may hang loose and flow downward from the top portion (i.e., be drapey).

In accordance with another aspect of the present disclosure and as shown in FIGS. 5A-5C, drape 10 may move from an undeployed configuration to an open, deployed configuration. In the undeployed configuration (FIGS. 5A and 5B), body 20 is substantially flat so that bottom edge 40 is substantially planar with top edge 35. Thus, in some embodiments using shaping elements, first shaping element 63 is also substantially planar with second shaping element 65 in the undeployed configuration. However, in the open, deployed configuration (FIG. 5C), bottom edge 40 is disposed below top edge 35 in a longitudinal direction of drape 10. The imaging device may be secured to connecting element 50 either before or after the drape is moved from the undeployed configuration to the open, deployed configuration. It is contemplated that the imaging drape may have various configurations when in an undeployed configuration. For example, the drape may be folded with a bottom edge of the drape under the top portion 30, such that a user must open or deploy the drape by pulling upward on connecting element 50. In some cases, it may be desirable to connect the imaging device to connecting element 50 of the undeployed drape, and then pull the imaging device upward to deploy drape 10 as it moves away from a support surface on which it was positioned.

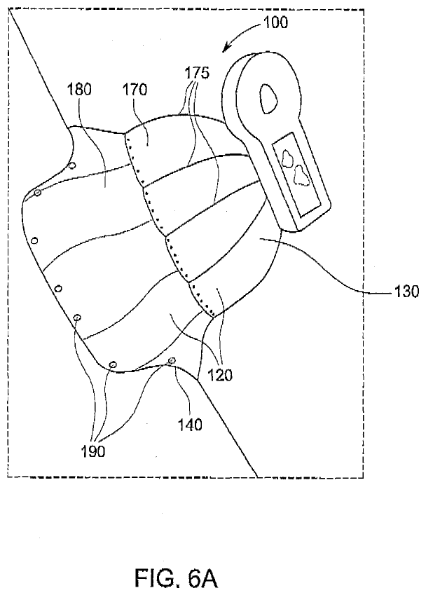

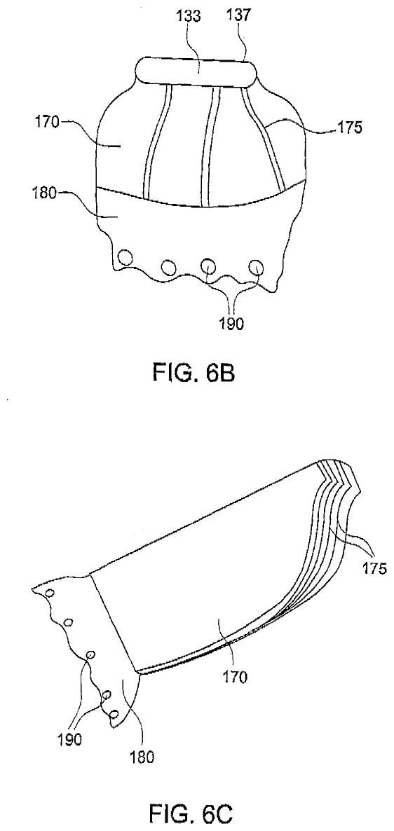

FIGS. 6A-6C show another example embodiment of an imaging drape, drape 100. Drape 100 includes a body 120 with a first drape portion 170 and a second drape portion 180. Similar to the other embodiments discussed herein, drape 100 includes a top portion 130 which includes a connecting element (not shown) defining an opening 133. Body 120 may extend from an outer edge or perimeter 137 of the connecting element to a bottom edge 140 of the drape 100. In the example embodiment of FIGS. 6A and 6B, top portion 130 of the drape is not defined by a top edge. Instead, top portion 130 slopes radially downward toward second drape portion 180, providing a smooth and continuous transition with the remainder of body 120 in second drape portion 180.

First drape portion 170 of drape 100 may be disposed above second drape portion 180 in the longitudinal direction of drape 100. In some embodiments, first drape portion 170 is a skirt portion or umbrella-like portion with a plurality of ribs 175 extending in a vertical direction of drape 100. Ribs 175 may be spaced apart from each other in a lateral direction of drape 100. The spacing may be consistent between each rib, or the spacing may be varied between the different ribs. Each rib 175 may be formed of a rigid material. The material of body 120 in first drape portion 170 may be disposed over or under ribs 175. Thus, in some embodiments, ribs 175 may be disposed on an outer surface of body 120. It is also contemplated that ribs 175 are embedded within the material of drape body 120.

Second drape portion 180 may be directly connected to a bottom edge portion of first drape portion 170. Second drape portion 180 may flow radially outward from first drape portion 170 so that a bottom edge of second portion 180 has a larger outer diameter than a bottom edge of first drape portion 170. A length of second drape portion 180 in the longitudinal direction of drape 100 may be greater than a length of first drape portion 170. It also may be the same length or shorter.

In some embodiments, body 120 of first drape portion 170 is formed of a different material than body 120 of second drape portion 180. First drape portion 170 may be formed of a relatively harder, firmer, or more rigid material than second drape portion 180. Alternatively, the ribs may pull the material taut in the first drape portion 170, giving first drape portion 170 a smoother appearance than second drape portion 180. In some embodiments, second drape portion 180 is formed of a soft, cloth-like material that is flexible and lightweight and easily drapes over an object as shown in FIG. 6A. It is also contemplated that the body of first drape portion 170 is formed of either a flexible or rigid material. Thus, the skirt of first drape portion 170, including ribs 175, may be flexible or rigid in the deployed state.

A bottom portion of second drape portion 180 may include one or more fixing elements 190 to aid in a bottom portion of the drape body closely adhering to the contours of an imaging target, for example a patient's body having an imaging target such as a wound. Fixing elements 190 include a weighted element, a magnet, a drawstring, tape, wire, a hook, or a loop closure. In the embodiment of FIGS. 6A-6C, fixing elements 190 are weights that are spaced across a bottom edge perimeter of second drape portion 180. Fixing elements 190 may help to secure drape 100 to a patient in order to reduce/prevent inadvertent movement of drape 100 with regard to the patient. Such may be helpful to reduce the amount of ambient light entering the interior of the drape when imaging a wound. In some embodiments, fixing elements 190 are magnets that are magnetically coupled with magnets (for example, magnetic stickers) placed on a patient.

It is also contemplated that any of the embodiments discussed herein may include fixing elements 190 on the drape body. Although referenced with regard to FIGS. 6A-6C, fixing elements 190 are not limited to this embodiment.

As discussed above, in the embodiment of FIGS. 6A-6C, drape 100 may include one or more shaping elements, which may be disposed at top and bottom regions of the drape.

Ribs 175 may extend in a substantially vertical direction, from the first shaping element to the top of the second drape portion 180. In other embodiments, ribs 175 may extend from opening 133 to the top of the second drape portion 180. The skirt of first drape portion 170 is held out of the field of view of the imaging device. This may be accomplished in various ways, for example, by use of a second shaping element (as discussed above) in a lower portion or hem of the skirt portion extending around a perimeter of the skirt portion, by use of vertical ribs 175, or by other means that provide tension in the skirt portion to keep it expanded away from a center portion of the darkened imaging environment.

In the undeployed configuration, ribs 175 may be compressed in an accordion-type manner. Thus, first portion 170 of drape 100 may be closed in a relatively flat configuration, as shown in FIG. 6C.

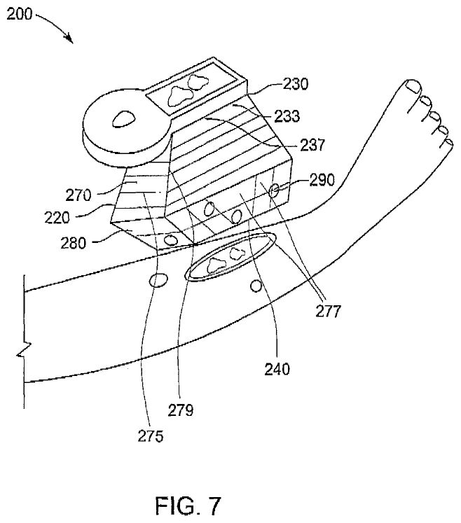

FIG. 7 shows yet another example embodiment of an imaging drape, drape 200. Drape 200 includes a body 220 with a first drape portion 270 and a second drape portion 280. Similar to the other embodiments discussed herein, drape 200 includes a top portion 230 with a connecting element (not shown) defining an opening 233 (not shown) within which the imaging portion of the imaging device may be positioned to view the imaging target. Body 220 extends from a perimeter 237 of the connecting element to a bottom edge 240 of the drape 200, In the embodiment of FIG. 7, top portion 230 is not defined by a top edge. Instead, top portion 230 slopes radially downward toward second drape portion 280, providing a substantially smooth and continuous transition with the remainder of body 220 in first drape portion 270 when the drape is in the expanded, deployed configuration. In some embodiments, the pleating in first portion 270 may give the first portion 270 more of a corrugated appearance than a smooth appearance.

First drape portion 270 of drape 200 may be disposed above second drape portion 280 in the longitudinal direction of drape 200. In some embodiments, first drape portion 270 has a bellows or accordion shape with a plurality of pleats 275 that fold upon themselves in the closed configuration. Pleats 275 may be formed in the material of first drape portion 270. Second drape portion 280 may extend longitudinally from first drape portion 270 towards bottom edge 240, Second drape portion 80 may be formed of a soft, cloth-like material that contours to the surface of the patient to be imaged. Thus, second drape portion 280 may not include the pleats. In some embodiments, first drape portion 270 is formed of a resilient plastic material and second drape portion 280 is formed of a soft plastic material.

In another example embodiment, instead of pleats a plurality of first ribs 275 extending in a horizontal direction of drape 200 may be provided in first drape portion 270. First ribs 275 may be spaced apart from each other in a longitudinal direction of drape 200, The spacing may be consistent between each rib, or the spacing may be varied between the different ribs. Each rib 275 may be formed of a rigid material. The material of body 220 in first drape portion 270 may be disposed over or under ribs 275. Thus, in some embodiments, ribs 275 may be disposed on an outer surface of body 220. It is also contemplated that ribs 275 are embedded within a material of drape body 220. In such an embodiment, the ribs may be planar with one another when the drape is in the collapsed or undeployed configuration, similar to what is shown in FIG. 5A, although the shape of the undeployed drape will be more rectangular rather than circular.

First drape portion 270 may also include one more second ribs 279 extending in a vertical direction of drape 200. Second ribs 279 may provide stability to ribs 275 so that the body of the drape does not inadvertently collapse.

In either example embodiment of the first drape portion 270, with pleats or with ribs, second drape portion 280 may include cloth type panels 277 configured to receive fixing elements 290, as discussed above. In one example, panels 277 may be folded over fixing elements 290 in order to secure fixing elements 290 on the drape.

Second drape portion 280 may be directly connected to a bottom edge portion of first drape portion 270. Second drape portion 180 may flow radially inward from first drape portion 270 so that a bottom edge of second drape portion 280 has a smaller outer diameter than a bottom edge of first drape portion 270. A length of second drape portion 280 in the longitudinal direction of drape 200 may be smaller than a length of first drape portion 270.

When moved from the open, deployed configuration to the undeployed configuration, pleats/ribs 275 fold upon themselves so that drape 200 assumes a substantially flat profile in the undeployed configuration. Thus, drape 200 is easily collapsible.

As discussed above, a bottom portion of second drape portion 280 may include one or more fixing elements 290 to aid in a bottom portion of the drape body closely adhering to the contours of a patient's body. Fixing elements 290 include a weighted element, a magnet, a drawstring, tape, wire, a hook, or a loop closure. In the embodiment of FIG. 7, fixing elements 290 are weights that are spaced across a bottom edge perimeter of second drape portion 280. Fixing elements 290 may help to secure drape 200 to a patient in order to reduce/prevent inadvertent movement of drape 200 with regard to the patient. Such may be helpful to reduce the amount of ambient light entering the interior of the drape when imaging a wound. In some embodiments, fixing elements 290 are magnets that are magnetically coupled with magnets (for example, magnetic stickers) placed on a patient.

It is also contemplated that any of the embodiments discussed herein may include fixing elements 290 on the drape body. Although referenced with regard to FIG. 7, fixing elements 290 are not limited to this embodiment.

As discussed above, in the embodiment of FIG. 7, drape 200 may also include one or more shaping elements, which may be disposed at top and bottom regions of the drape.

In accordance with another aspect of the present disclosure, an imaging device (such as the imaging device of FIG. 1 or FIGS. 120-16D) may be disposed on or partially within opening 33 of the connecting element so that the lens portion/imaging head/optical head is positioned to view the imaging target within the drape interior (the portable imaging environment).

With the imaging device connected to the drape, the drape is positioned so that the interior of the drape at least partially surrounds or encompasses a surface on which the imaging target is disposed. For example, if the imaging target is a wound on a bottom of a foot, the drape may be positioned to encompass the foot. Alternatively, if for example the target is a wound on a leg, as shown in FIG. 7, the drape is positioned to surround the surface on which the target is positioned. The drape may be secured to that surface in order to minimize any ambient light entering the portable imaging environment created by the drape and, in which the target is positioned.

After positioning the drape, the imaging device, with the imaging head/optical head that includes at least the lens and the excitation light source positioned to have in its field of view (FOV) the target within the portable imaging environment, is actuated to emit excitation light to illuminate the target, such as a wound. If fluorescent imaging is being performed, the drape used is a darkening drape configured to provide a darkened imaging environment with reduced or limited ambient light. Thus, the drape may reduce/prevent ambient light from entering the interior environment of the drape. The inventors have found that, to provide acceptable quality fluorescent images, the amount of ambient (visible) light in the imaging environment should be less than about 402 mW/m2. In some embodiments, the drape may be configured to provide an imaging environment having less than about 402 mW/m2 of ambient light. Thus, the inventors have established a threshold/acceptable level of ambient light for fluorescent imaging at about 402 mW/m2 of ambient light. In other embodiments, the drape may be configured to provide a portable imaging environment having less than about 401.1 mW/m2, less than about 350 mW/m2, less than about 300 mW/m2, less than about 250 mW/m2, less than about 200 mW/m2, less than about 190 mW/m2, and/or less than about 185 mW/m2 of ambient light. In one example embodiment, fluorescent imaging was performed in a well-lit hospital room. The amount of ambient light in the hospital room was measured to be 290,200 mW/m2. The amount of ambient light measured in the portable darkened imaging environment formed by a drape in accordance with the present disclosure was 184.6 mW/m2. Thus, in this example, the imaging drape prevented about 99.94% of ambient light in the external imaging environment from entering the portable imaging environment defined by the interior of the imaging drape.

As discussed above, connecting element 50 is configured to secure the imaging device to the drape during an imaging procedure. Connecting element 50 may be attached to the drape via welding, adhesive, or any other well-known attachment means. It is also contemplated that connecting element 50 and drape 10 are formed as a unitary member, Connecting element 50 may be removably attached or permanently attached to the drape. In some embodiments, connecting element 50 is formed of an injection molded plastic material, such as, for example, high density polyethylene.

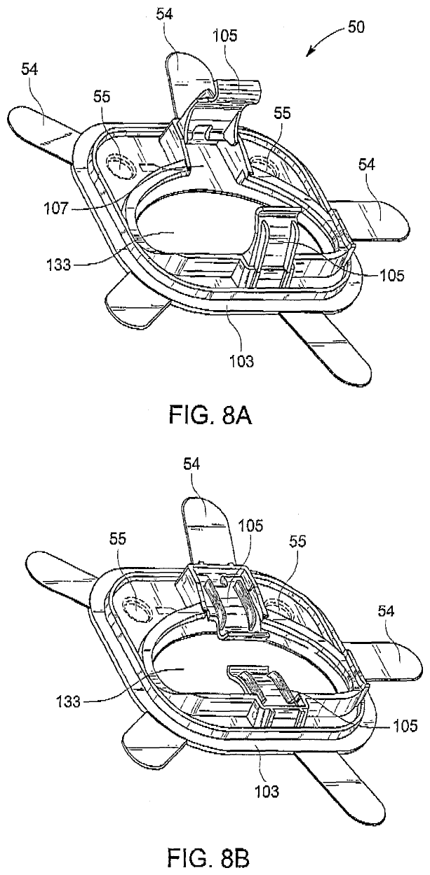

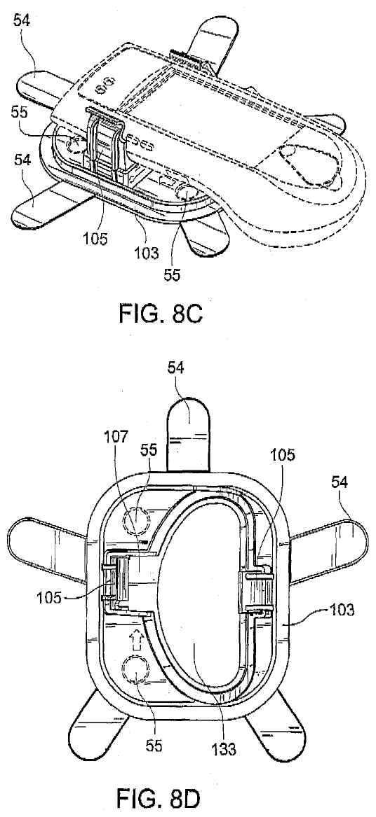

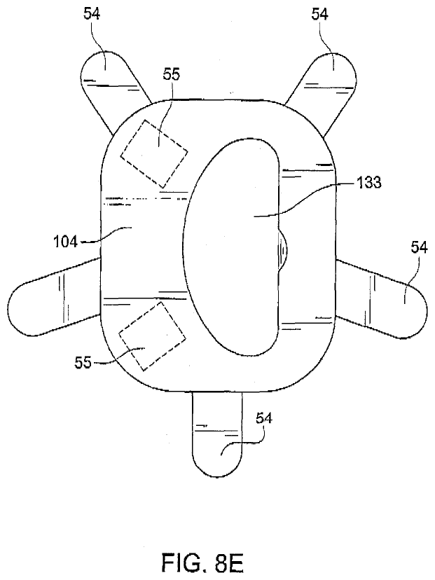

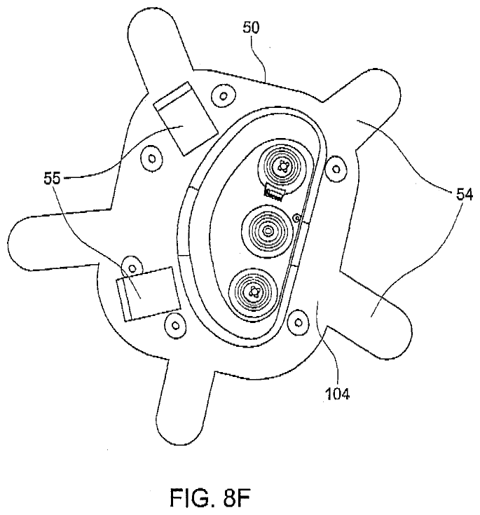

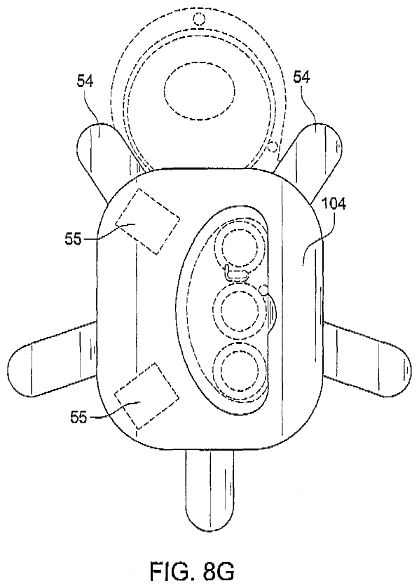

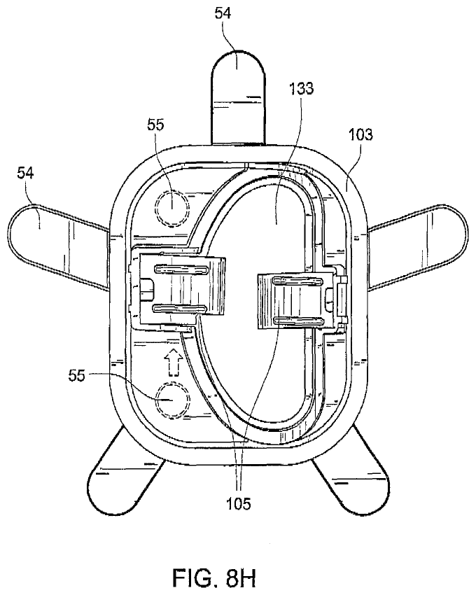

As shown in the example embodiment of FIG. 8A, connecting element 50 forms a frame around opening 133, which is the opening into the drape body and the opening through which the imaging device “views” the imaging target and images the target. The frame may be substantially rectangular in shape and includes a top, planar surface 103 and one or more grasping elements configured to “hold” the imaging device. The grasping elements may be referred to herein as clasps, clamps, or clips, Grasping elements 105 (e.g., connectors) project outward from surface 103 when grasping elements 105 are in an open configuration. Grasping elements are configured to rotate in a range of motion of about 90 degrees from the open configuration (FIG. 8A) to a closed configuration (FIG. 8B). When in the open configuration, grasping elements 105 may have a slight bias inward, toward a center of connecting element 50, so that the imaging device may be secured on top, planar surface 103 of connecting element 50 between grasping elements 105. Thus, grasping elements 105 secure the imaging device using a snap-fit arrangement. However, it is also contemplated that the imaging device may be secured to connecting element 50 using, for example, clips, straps, tape, Velcro, adhesives, buttons, or any other well-known attachment means. FIG. 8C shows a view of the imaging device secured within grasping elements 105.

In the closed configuration, as shown in FIG. 8B, grasping elements 105 are folded down so that they are flush with surface 103. For example, grasping elements 105 may fit within recesses 107 when folded down in order to minimize their vertical extension from surface 103 in the closed configuration. Such may help to minimize height and space requirements during packaging.

As shown in FIGS. 8A and 88, planar surface 103 forms opening 133 which may be substantially kidney-shaped. When an imaging device is disposed in connecting element 50, a lens and an excitation light source of the imaging device are aligned with opening 133 so that the FL light from the imaging device is emitted through opening 133. Additionally, the opening 133 is sufficiently sized so that the body of the imaging device blocks the top of the opening and ambient light does not enter the interior of the drape environment from this opening. Thus, opening 133 of connecting element 50 is configured to sealingly engage the imaging device to prevent passage of electromagnetic radiation through the opening and into an interior of the drape.

FIGS. 8A-8D show a top view of connecting element 50. FIG. 8E shows a bottom side of connecting element, which is disposed on a bottom, interior portion of the drape when connecting element 50 is attached to the drape. Bottom side of connecting element 50 includes a bottom, planar surface 104 and one or more projections 54 extending from surface 104. Projections 54 may be spaced apart to give structure to the drape to prevent the material of the drape from collapsing into the field of view. Projections 54 are also flexible and able to bend to allow for imaging near surfaces such as walls or beds. Planar surface 104 and projections 54 may also prevent/reduce any light from entering the interior of the drape through the connection between the drape and the imaging device.

An underside of each of planar surfaces 103, 104 may include an adhesive material for attachment to the drape. Thus, a back side of these surfaces may comprise an adhesive backing. However, it is also contemplated that planar surfaces 103, 104 may each be attached to the drape through any well-known attachment means such as welding, Velcro, staples, and/or tape. Additionally, as discussed further below, a valve 55 may be disposed through planar surfaces 103, 104 to aid in deployment of the drape,

FIGS. 8F and 8G show the bottom side of connecting element 50 and the drape with an imaging device disposed within opening 133. As discussed further below, a lens and the excitation fight source(s) of the imaging device are disposed within opening 133 in order to perform FL imaging within the portable imaging environment of the drape. A white-light source, such as a flash, may also be disposed on the portion of the imaging device disposed within opening 133.

It is also contemplated that other connecting elements may be used to attach the imaging device to the drape, including, for example, clips, hooks, and loop fasteners. These other connecting elements may be used separately from or incorporated with connecting element 50. FIG. 8H shows another example embodiment of connecting element 50 in which grasping elements 105 are offset from each other. Such may be used to decrease any inadvertent movement of the imaging device when secured on connecting element 50.

FIG. 8I shows an embodiment in which the connecting element 50 is attached to a mask 700, which may be formed of, for example, a polyethylene film. Mask 700 may include an adhesive backing that attaches to surface 104 of connecting element 50. Mask 700 may prevent/reduce light leakage from entering through the connection point between the imaging device and the drape. Mask 700 may include one or more through-holes 720 that are configured to be aligned with flap valve 55, as discussed below.

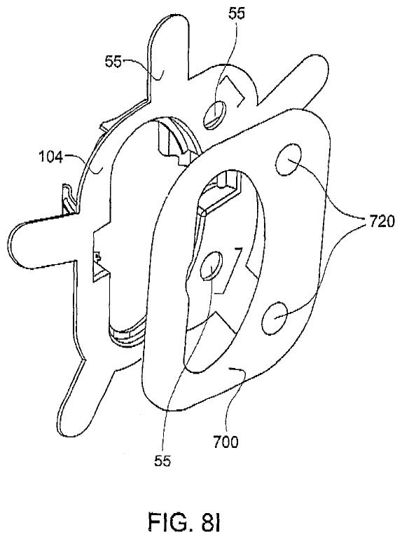

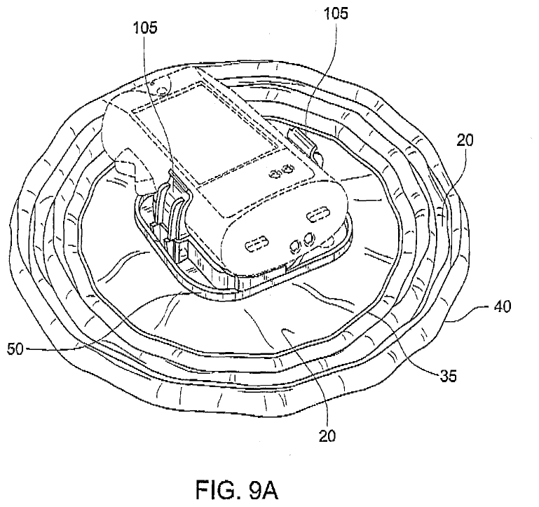

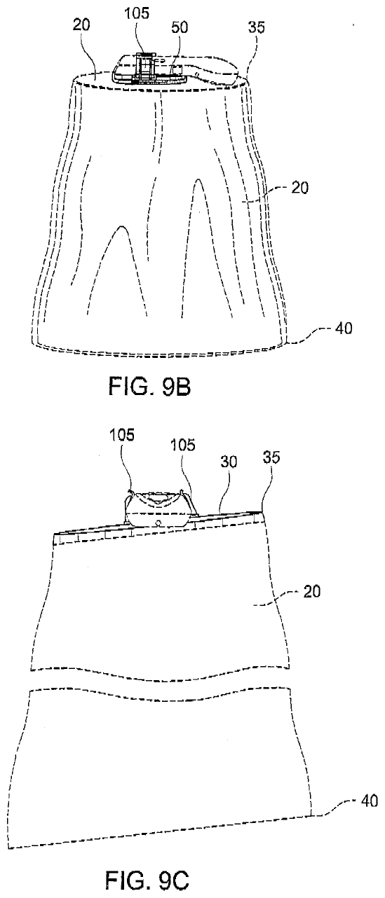

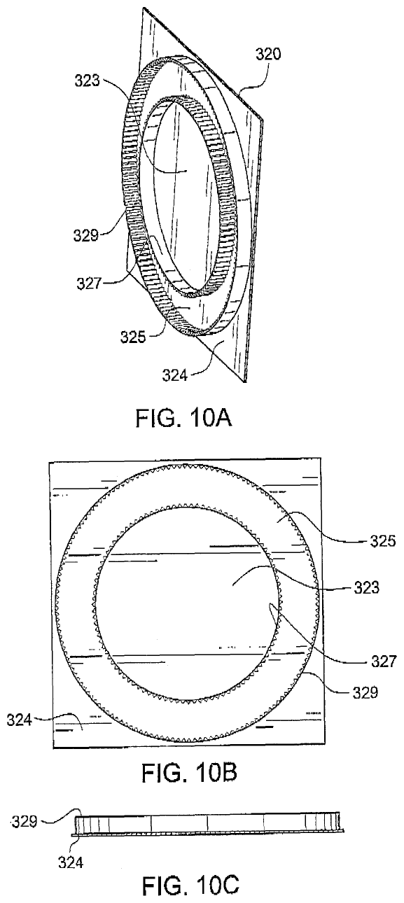

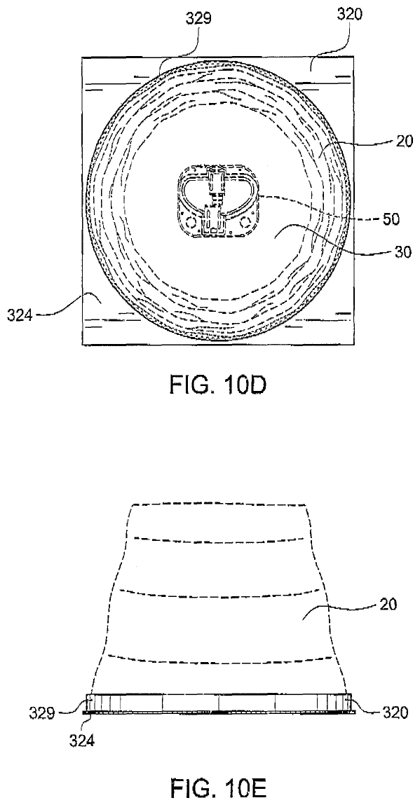

As discussed above, the imaging device may be secured to the drape either before or after the drape is deployed. FIG. 9A shows the imaging device secured to the drape in its undeployed configuration. FIGS. 9B and 9C show the imaging device secured to the drape in its open, deployed configuration. In the undeployed configuration, the drape may lay substantially flat. The drape is substantially flexible in both configurations. As shown, for example, in FIG. 9A, the drape may have a circular shape when undeployed. However, it is also contemplated that the drape may assume a variety of shapes in the undeployed and deployed configurations. In accordance with one aspect of the present disclosure, the drape may be stored in a sterilized condition before use. The drape may be stored, for example, on a dispenser device 320 such as the device shown in FIGS. 10A-10C. FIG. 10D shows an image of an example drape in an undeployed condition on dispenser device 320. As shown in FIGS. 10A-10C, dispenser device 320 includes first and second cavities 323, 325 separated by first wall 327 on planar surface 324. A second wall 329 forms a border along the outer perimeter of second cavity 325. First and second walls 327, 329 may form restraining elements and may each have a height that extends upwardly from planar surface 324 of dispenser device 320. FIGS. 10A-10C shows walls 327, 329 as being concentrically positioned and as having a circular shape. However, it is contemplated that the walls may include any shape, as is well-known in the art. In some embodiments, the walls have the same shape as shaping elements 60.

When the drape is in the undeployed configuration, as shown in FIG. 10D, cavities 323, 325 are configured to receive the drape body so that the material of the drape does not become tangled, which may help to provide an even deployment of the drape. Additionally, first and second walls 327, 329 may form barriers that are configured to engage shaping elements 60 of the drape. For example, shaping elements 60 may be disposed between walls 327, 329 and within cavities 323, 325 of dispenser device 320, when the drape is in the closed, undeployed configuration.

Walls 327, 329 and shaping elements 60 are configured to cooperate together to facilitate deployment of the drape. FIG. 10E shows the drape after deployment from dispenser device 320.

Dispenser device 320 may be formed of, for example, cardboard, plastic, or rubber. The interior of the drape may be maintained in a sterile condition against dispenser device 320 when the drape in the closed, undeployed configuration.

In one example embodiment, one or more one-way valves, such as flap valves 55 on connecting element 50, may assist in deployment of the drape from dispenser device 320. As the drape is moved from the closed or undeployed configuration to the open or deployed configuration, for example, as it is pulled away from dispenser device 320, air may move from outside the drape to within the interior of the drape through flap valves 55. Such air movement into the interior of drape creates a higher air pressure within the drape than exterior of the drape, thus helping to maintain and hold the drape in the open, deployed configuration. During imaging, flap valves 55 remain closed. Flap valves 55 may be formed of the same material as the material of drape body 20. Each flap valve 55 may be a piece of material that is secured to connecting element 50 over a hole in connecting element 50. Although described with reference to dispenser device 320, flap valves 55 may also assist in deploying the drape when dispenser device 320 is not used, for example, when the drape is opened from a closed configuration on a user's working desk.

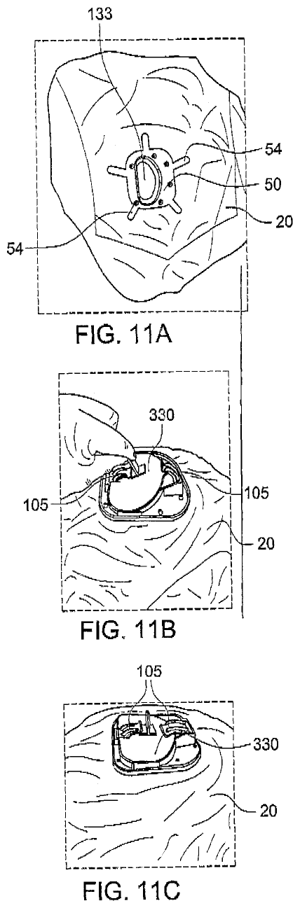

As shown in FIGS. 11A-11C, when stored in a sterilized condition, the drape may be provided with a sterile cover 330. The cover 330 is positioned between, for example, planar surface 103 of connecting element 50 and grasping elements 105 and is positioned over opening 133. Removing the sterile cover 330 causes grasping elements 105 to open from a closed storage position to an open position to receive the handheld imaging device and to expose the opening 133 to receive the lens portion of the imaging device. Sterile cover 330 may have substantially the same shape as the opening 133 in connecting element 50.

FIG. 11C shows sterile cover 330 positioned on drape, FIG. 11B shows sterile cover 330 as it is being pulled upward and removed from the drape, and FIG. 11A shows the drape with sterile cover 330 removed.

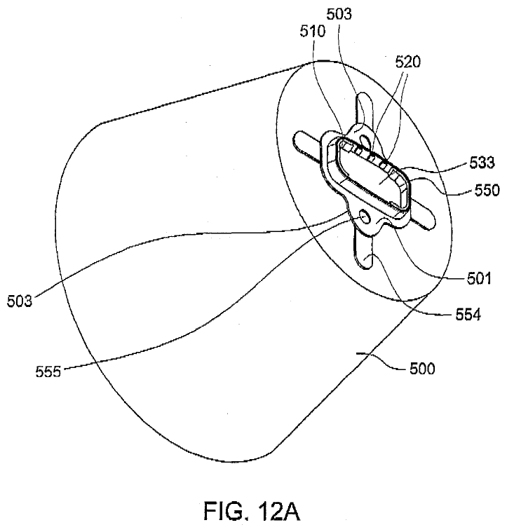

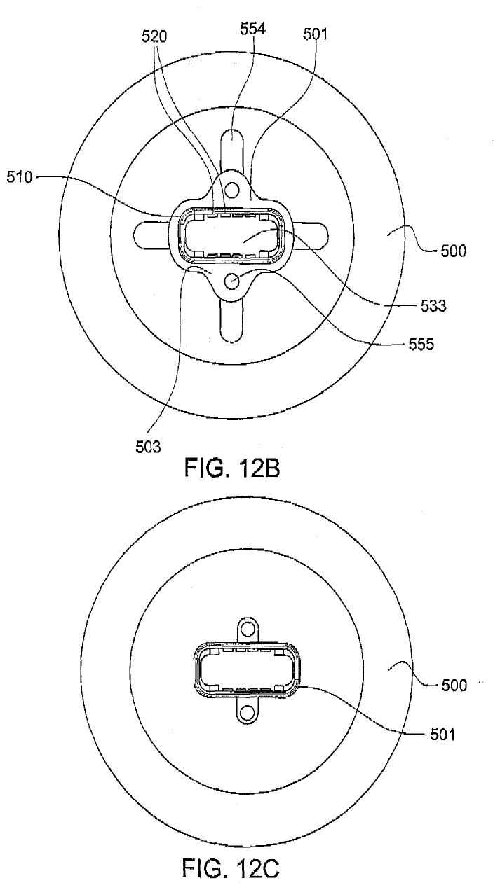

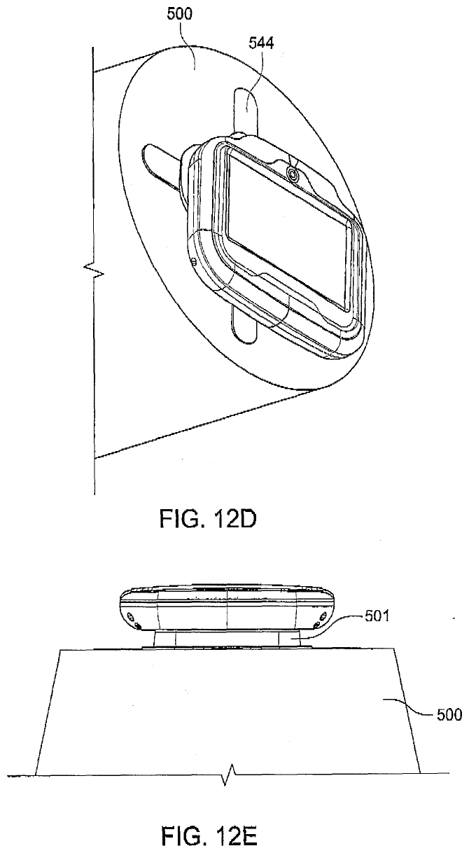

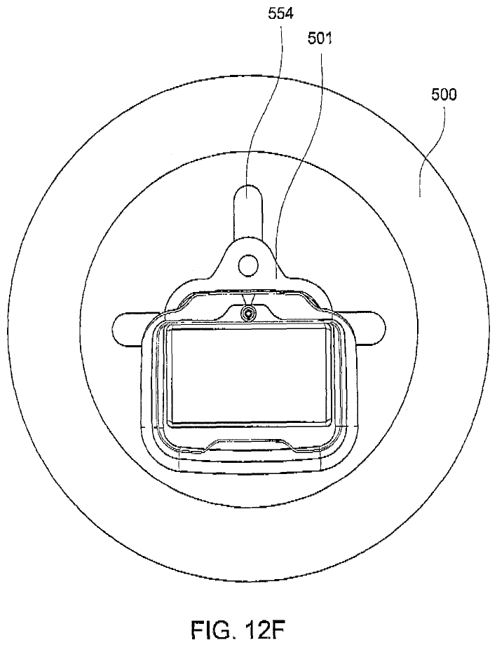

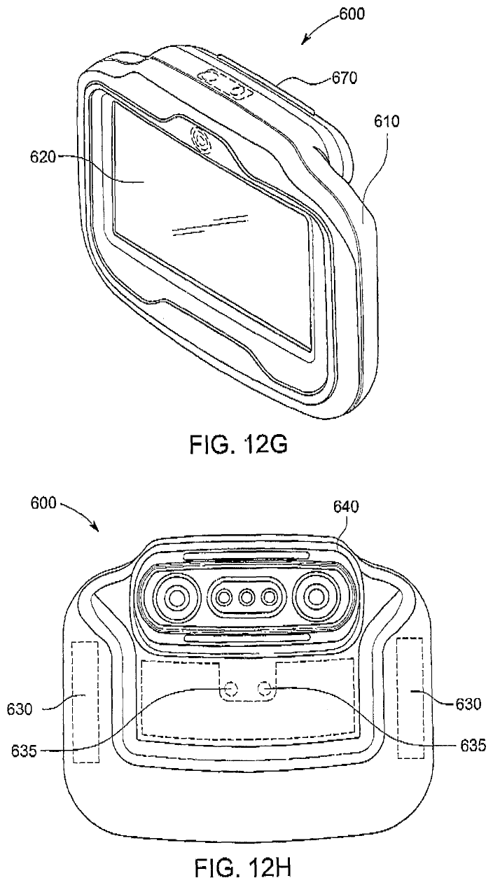

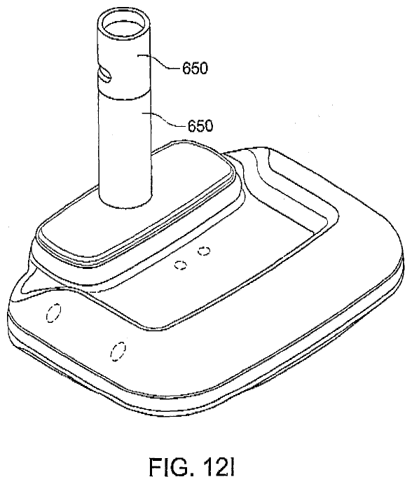

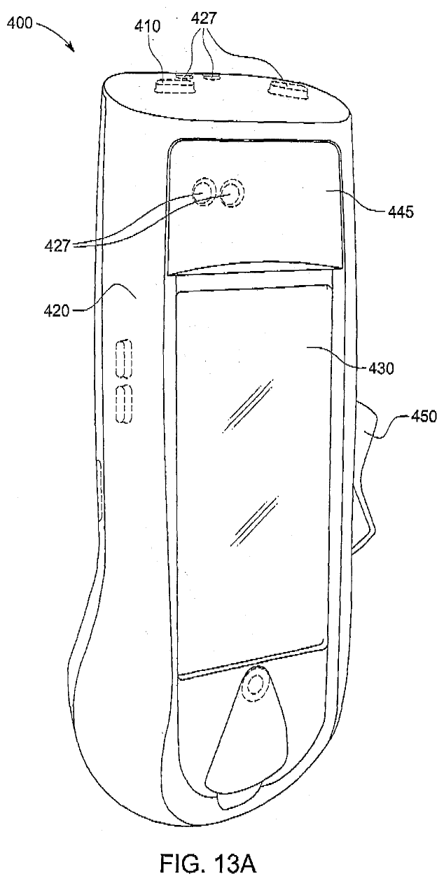

FIGS. 12A-12C show another example embodiment of an imaging drape 500. The imaging drape 500 is shown connected to an imaging device in FIGS. 12D-12F. Drape 500 includes connecting element 501. In the example embodiment, connecting element 501 does not include grasping elements, as discussed above, to assist in securing the imaging device to the drape. Instead, connecting element 501 includes a ridge 510 that is used to form a press-fit or snap-fit connection with the imaging device. A projection on the imaging device (as discussed below) may engage with ridge 510 to provide the press-fit or snap-fit connection. Additionally, one or more protrusions 520 may be configured to engage with the projection on the imaging device to grip the imaging device and better secure it to the drape. In some embodiments, protrusions 520 are teeth-like members that engage with the projection on the imaging device. When an imaging device is properly aligned and pressed down into connecting element, protrusions 520 clip into the projection on the imaging device in order to provide the snap-fit connection.

Connecting element 501 may also include end cup members to 550 to help facilitate the snap-fit connection between connecting element 501 and the imaging device. As shown in FIG. 12A, end cup members 550 may be smooth members disposed at either end of opening 533, which is formed within connecting element 501. End cup members 550 may provide guidance to center/position the imaging head/optical head of the imaging device that is snap-fitted into connecting element 501.

FIG. 12A shows opening 533 as having a rectangular shape with protrusions 520 disposed on the longer sides of the rectangular shape and end cup members 550 disposed on the shorter sides of the rectangular shape. However, it is also contemplated that protrusions 520 may be disposed on the shorter sides of the rectangular shape and that end cup members 550 may be disposed on the longer sides of the rectangular shape. Although FIG. 12A shows two end cup members 550, only one end cup member 550 may be used on one side of opening 533. Furthermore, in some embodiments, connecting element 501 may not include end cup members 550. In this embodiment, protrusions 520 may be disposed around a majority or the entire perimeter of opening 533 on connecting element 501. Connecting element 501 may be formed of injection molded plastic, as discussed above.

FIG. 12A shows a top, perspective view of connecting element 501 secured to a drape. FIG. 12B shows a top view of connecting element 501 and an exterior view of the drape, and FIG. 120 shows a bottom view of connecting element 500 and a view of the portable imaging environment formed by an interior of the drape.

Similar to the embodiments discussed above, connecting element 501 may also include a top, planar surface 503, one-way valves, such as a flap valves 555, and projections 554. However, in the embodiment of FIGS. 12A-12F, projections 554 are disposed on a top surface of connecting element 501. Therefore, projections 554 are viewable from the top view of FIG. 12B but are not viewable from the interior of the drape in the view of FIG. 12C. Projections 554 help to hold the drape material out of the imaging field of view, as discussed above.

In the embodiment of FIGS. 12A-12F, connecting element 501 may be formed of an injection molded plastic, such as polyethylene, Thus, connecting element 501 may be a relatively stiff member. In some embodiments, connecting element 501 has a thickness of about 1.8 mm, Projections 554 may be formed of the same material as the remainder of connecting element 501, but may be less stiff than the remainder of connecting element 501. Thus, projections 554 may be thinner than the remainder of connecting element 501. The material of the drape body may be formed of the same material as connecting element 501, but may not be ejection molded so that the material of the drape body is less stiff than connecting element 501 (including projections 554). In some embodiments, the material of the drape body is also thinner than connecting element 501 (including projections 554). The drape body may be formed of a soft material that is welded to the relatively stiffer material of connecting element 501. This may lower manufacturing costs by allowing flap valves 555 to be integrated into the drape by being formed by the material of the drape body.

Similar to the embodiments discussed above, connecting element 501 also includes opening 533 in order to provide, from the imaging device, FL and/or white-light imaging within the interior environment of the drape. In the embodiment of FIGS. 12A-12C, opening 133 is substantially rectangular in shape, as discussed above. However, it is further contemplated that other shapes may be used.

FIGS. 12D-12F show an example of an imaging device 600 secured to connecting element 501 of the drape shown in FIGS. 12A-12C. As discussed above, the imaging device 600 is securely fastened to connecting element 501 through a snap-fit connection that prevents/reduces any ambient light from entering the interior of the drape through the top of the drape. A projection 670 on imaging device 600 may engage with protrusions 520 and ridge 510 on connecting element 501 to provide the snap-fit connection, as discussed above.

An example embodiment of a modular handheld imaging device 600 is shown in FIGS. 12G and 12H. Imaging device 600 includes a base body portion 610 with a generally square or rectangular shape. A front, or user-facing side of the base body portion 610 includes a display screen 620 for displaying images and videos captured by the device. Projection 670 projects outward from optical head/optical housing 640 although, alternatively, it may be positioned on base body portion 610, Although FIG. 12G shows projection 670 as disposed on a top portion of base body portion 610, it is also contemplated that projection 670 may be disposed on other sides of base body portion 610, depending on the location of protrusions 520 on connecting element 501.

Although depicted as square or rectangular, imaging device 600 may take on any shape that will reasonably support a display screen such as a touchscreen. In addition to disclosing images captured by the imaging device 600, the display screen also operates as a user interface, allowing the user to control functions of the device via touchscreen input.

Positioned on an opposite side of the device, a patient-facing side of the device, may be handhold areas 630 configured to facilitate a user holding the device during imaging. The patient facing-side of the device may also incorporate contacts 635 for wireless charging of the device.

In accordance with one aspect of the present disclosure, the patient-facing side of device 600 also includes an optical housing 640. Optical housing 640 may be detachable from base body portion 610. Optical housing portion 640 is illustrated as a rectangular housing configured to be received in the opening of the connecting element on the drape.

The optical housing 640 may take on different configurations. For example, as shown in FIG. 12H, the optical housing portion 640 has a generally flat, oblong shape. Optical components, for FL and/or white light imaging, are arranged in a generally linear manner across a width of the optical housing. The optical components are described in greater detail below.

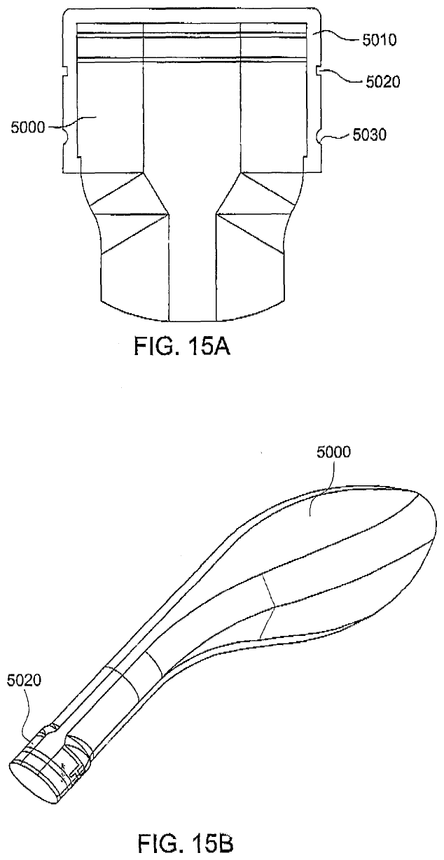

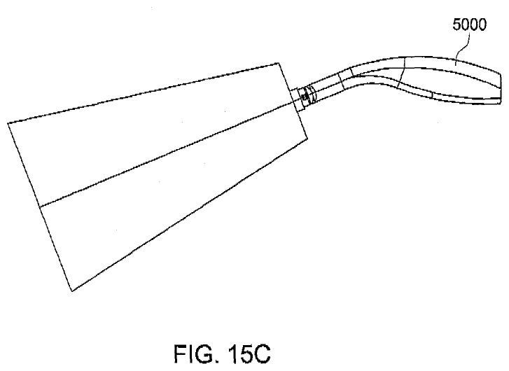

In some embodiments, as shown in FIG. 12I, the optical components may be disposed in a distal tip 660 of an endoscope 650. Thus, endoscope 650 may be disposed within the opening of the connecting element to direct the FL and/or white-light image onto the area of the patient to be imaged. When connecting with the optical portion of endoscope 650, the drape connector 501 may have a circular shape. Additionally or alternatively, the endoscope 650 may be provided with a shield similar to the one discussed below with regard to FIGS. 15A-15C, which may then connect to a portable imaging environment in accordance with the present disclosure.

It is within the scope of this disclosure that various aspects of the above-discussed embodiments of the drape may be combined, Thus, the features of each of these embodiments may be combined with features of the other embodiments. Various features of the different embodiments are not mutually exclusive and, instead, can be combinable as those having ordinary skill in the art would understand. An example of deployment and use of the drape, with the MolecuLight i:X imaging device is described below. The method may include obtaining a fluorescence-based image or video of a target portion of a patient without changing ambient light conditions. The drape is first removed from its sterile packaging. The operator may then remove sterile cover 330 on connecting element 50. Removal of sterile cover 330 causes the grasping elements 105 to open and project upward. The imaging device is then attached to the drape by being clipped into grasping elements 105 of connecting element 50. The lens of the imaging device is fitted into opening 133 on connecting element 50. The operator may then lift the imaging device upward and away from dispenser device 320, thus deploying the drape in one simple motion as air moves through valves 55,

Once the drape is deployed, the operator may position the drape over the area of the patient to be imaged. The body of the drape may be positioned to nest within the different contours of the patient's body. In some embodiments, fixing elements 190 may be coupled to magnetic stickers on the patient's body, in order to provide proper alignment with the drape and the contours of the patient's body. It is also contemplated that the operator may bend or otherwise manipulate shaping elements 60 so that the drape better nests within the contours of the patient's body.

Next, the operator may ensure that the proper imaging conditions are met, for example, that the interior environment of the drape is sufficiently dark. If the drape body is not properly nested with the contours of the patient's body, the interior environment of the drape may not be sufficiently dark. The operator may verify that the ambient light sensor of the imaging device shows that the proper imaging conditions are met. In some embodiments, the ambient light sensor may display green when the proper imaging conditions are met.

The operator may turn on the violet LEDs and capture a FL image or video using the drape. Additionally or alternatively, the operator may capture an image or video with the drape using white-light imaging. Upon completion of the imaging, the drape is removed from the patient and from the imaging device. The drape may then be disposed of or sterilized for re-use.

The drape may be manufactured according to the exemplary method discussed below. The material forming the body 20 of the drape may be cut into the shape of an arc and then formed into, for example, a truncated cone shape (an outer arc length of the arc forming the bottom circumference of the cone and an inner arc length of the arc forming the top circumference of the cone). Additionally, the material forming the body 20 of the drape may be cut into a circular shape with an opening for attachment to connecting element 50/501. The circular shape may then be welded to the truncated cone shape to form the drape.

The connecting element 50/501 may be formed by injection molding. The connecting element 50/501 may be welded to the material of the drape body, for example by plastic welding. Although it is contemplated that other mounting methods may be used. In some embodiments, the body of the drape and the connecting element 50/501 may be unitarily formed, for example, in embodiments where these components are formed of molded rubber. The metal wires forming the shaping elements may be embedded in the drape by tapping or welding.

The drape is manufactured and packaged in such a way that the end-user will receive a sterile product. The drape may also be deployed from its packaging by the operator without compromising the sterility of the inside wall of the drape (the portion of the drape contacting the patient).

An exemplary method of obtaining a measurement of a target without changing ambient light includes removing cover 330 from an opening of the drape. Next, an operator may attach an imaging device to the drape while the drape is connected to a packaging element, such as dispenser device 320. Then, the operator may move the imaging device and attached drape away from the packing element to deploy the drape. The operator may position an open, bottom end of the drape over an area of a body containing the target to be measured. A portion of the drape around the body area containing the target to be imaged may be contoured to create a darkened environment around the body area containing the target to be measured, such that the drape is configured to limit the passage of electromagnetic radiation through the drape into the darkened environment. An operator may also measure the target on the body area with the imaging device attached to the drape. As discussed further above, the target area may be a wound, lesion, cut, incision, or tumor. When FL imaging a wound, the clinician may be intent on visualizing any bacteria in the wound. During FL imaging of bacteria, when illuminated by blue/violet excitation light, for example excitation light having a wavelength of 405 nm±10 nm, the bacteria will emit fluorescence signals responsive to the illumination. The signals may have wavelengths between about 500 nm and about 550 nm and greater than about 600 nm, in some cases between about 600 nm and about 660 nm. A filter in the imaging device is configured to allow signal corresponding to this autofluorescence of the bacteria to pass through the filter to an image sensor of the imaging device.

The operator may measure the target on the body area by capturing and viewing a white-light image of the target on a screen on the imaging device. The white-light image may be captured in the darkened environment using the drape. Additionally or alternatively, the operator may measure the target on the body area by capturing and viewing a FL image of the target on a screen on the imaging device. The FL image may be captured in the darkened environment using the drape.

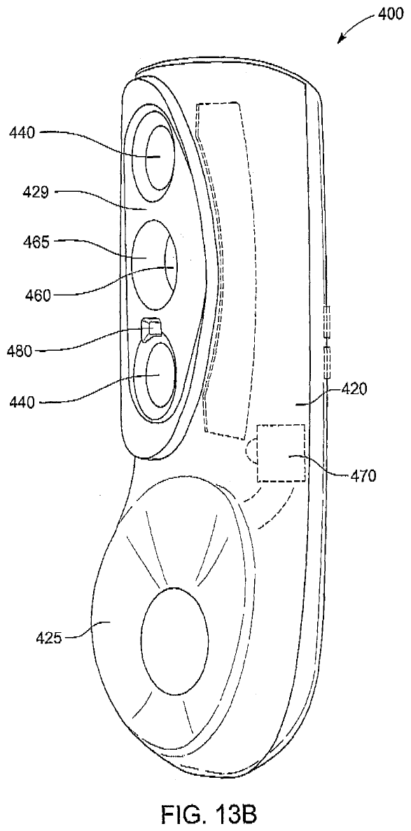

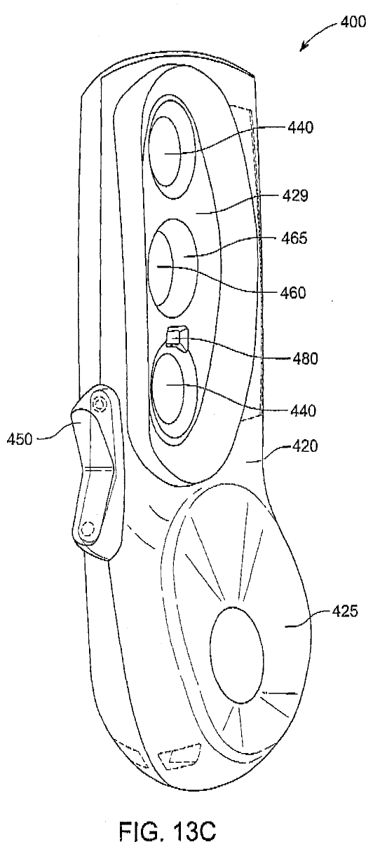

FIGS. 13A-13C show an exemplary imaging device 400 that may be used with some of the embodiments of the drape disclosed herein. Imaging device 400 is a portable, handheld device that does not require contact with a patient to obtain a white-light or FL image/video of a target area on the patient. As discussed above, in some examples, the target is a wound on the patient. An operator of imaging device 400 is not required to wear eye or skin protection when operating the device. Imaging device 400 may be used to capture real-time images/videos. The device includes a pulsed laser-base range finder to determine the optimal distance between the device and target area for superior image quality. The device does not require the use of exogenous imaging contrast agents to obtain the FL image/video.

As discussed below, imaging device 400 includes a portion with a lens and at least one violet light source. Connecting element 50/501 is configured to encompass an area surrounding the lens and the at least one light source to position the light source within the interior space of the drape body.

As shown in FIG. 13A, imaging device 400 includes a power button 410 and a display screen 430 on a body 420. Heat sink 445 is located on body 420 and is used to dissipate heat during use of the device.

As shown on the rear side of imaging device 400 in FIG. 13B, body 420 includes an illumination zone 429 comprising one or more excitation light sources, LEDs 440, to provide illumination when the device is in FL imaging mode. In some embodiments, illumination zone 429 includes two violet LEDs configurated to provide excitation light at about 405 nm. During FL imaging, the excitation light is directed to and illuminate an imaging target, such as for example, a wound and surrounding skin, for high-resolution and real-time fluorescence imaging of bacteria and tissue components contained within the wound. Illumination with the excitation light causes bacteria and tissue components within the wound to fluoresce, without the need for contrast agents, and the imaging device captures the fluorescent signals emitted by the bacteria and tissue components. When the wound is illuminated by the 405 nm excitation light emitted, for example, from LEDs 440, collagens in the connective tissue matrix emit a green colored fluorescent signal, and some bacteria emit a red colored fluorescence signal due to the production of endogenous porphyrins, while other bacteria emit a cyan colored fluorescence signal due to the production of endogenous pyoverdine. Imaging device 400 may simultaneously capture fluorescence from bacteria and other tissues and create a composite image on display screen 430. The user can easily and instantly visualize the presence and location of bacteria within and around the wound on the patient.

LEDs 440 are configured to produce excitation light to illuminate a target in a patient to elicit an optical signal (e.g., fluorescence) to be imaged with, for example, violet or blue light (e.g., 400-450 nm), or any other combination of single or multiple wavelengths (e.g., wavelengths in the ultraviolet/visible/near infrared/infrared ranges), LEDs 440 are configured to produce light having wavelengths of between about 400 nm and about 450 nm, about 450 nm to about 500 nm, about 500 nm to about 550 nm, about 600 nm to about 650 nm, about 650 nm to about 700 nm, about 700 nm to about 750 nm, and about 750 nm to about 900 nm. In some embodiments, LEDs 440 are an LED array, a laser diode, and/or filtered lights in a variety of geometries.

As shown in FIG. 13B, illumination zone 429 may also comprise a lens that directs the fluorescence signals emitted by the target to a filter, and then to an image sensor contained in the body of the imaging device. The image sensor provides image and video capture of both fluorescent and white light signals. Illumination zone 429 (also known as the imaging head or optical head) may include a flash for white light imaging. Illumination zone 429 is disposed within the opening in the connecting element of the imaging drape in a press-fit or snap-fit manner and may be further secured by clamps, clips, or other fastening elements, as discussed above. When positioned within the opening of the drape via the connecting element, the lens of the illumination zone can direct fluorescent signals emitted by the imaging target to the image sensor to capture white-light and/or FL image/video.

Body 420 of imaging device 400 may include a switch 450 to toggle between white-light and FL imaging modes. Body 420 may also include holding contours 425 that allow a user to easily grip the device during use. In some embodiments, body 420 may have a charging port 470, which allows the device to be charged with a power cable.

Imaging device 400 may also include one or more sensors 480, for example a range finder sensor to detect an optimal distance from the wound and an ambient light sensor to detect optimal lighting conditions for FL imaging mode.

A front side of imaging device 400 may include status indicators 427 to indicate an overall device performance status, a battery charge status, an optimal distance from the wound, and an optimal lighting environment for FL imaging mode.

Imaging device 400 may also include one or more optical filters (not shown) to remove any undesired wavelengths. Additionally, imaging device 400 may include a rechargeable battery (not shown).

The imaging device may be used to instantly visualize the presence of potentially harmful bacteria commonly found within or around a wound, including S. aureus, P. aeruginosa, E. coli, Coagulase-negative staphylococci, Enterococcus spp, Proteus spp, Klebsiella pneumoniae, Beta-hemolytic streptococci (Group B), and Enterobacter app. The bacteria may be visualized by imaging device 400 during clinical assessment, treatment, and/or monitoring of the treatment of response of the wound.

As discussed above, imaging device 400 may be used with the drape disclosed herein to obtain a white-light and/or FL image/video. During use, imaging device 400 should be held about 8-12 cm away from the wound. Thus, the device does not require contact with the patient during operation. The range finder sensor may be green when the distance between the device and the wound is optimal. Additionally, imaging device 400 should be held so that a plane of the wound is approximately parallel to a plane of the device.

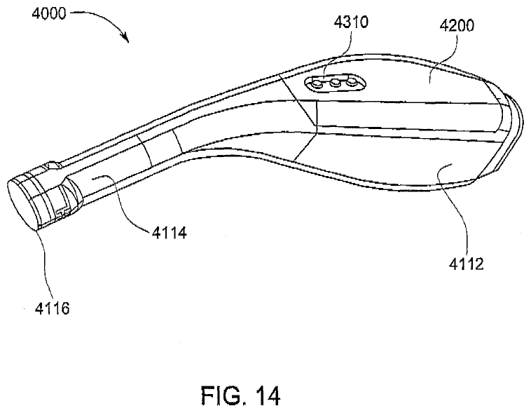

The various components of imaging device 400 may be incorporated into the other imaging devices disclosed herein, such as the imaging devices of FIGS. 12G, 12H, 12I, and 14.

FIG. 14 shows another embodiment of an imaging device that may be used with the various exemplary embodiments of the drape disclosed herein. Imaging device 4000 includes a body 4200 having a first end portion 4112 and a second end portion 4114. The first end portion 4112 is sized and shaped to be held in a single hand by a user of the device. The first end portion 4112 may include controls 4130 configured to actuate the device, toggle between and/or otherwise control different light sources, and switch between one or more optical imaging filters. Such controls can include buttons, switches, capacitive discharge sensors, or other devices to be manipulated by the user.

Second end portion 4114 of imaging device 4000 may be tapered and/or elongated to facilitate insertion of a distal end or tip 4116 of the second end portion into a surgical incision of 2-3 cm in size and into a patient's surgical cavity from which a tumor or cancerous tissue has been removed, for example, during breast cancer surgery. Second end portion 4114 may be rigid and positioned at an angle relative to first end portion 4112 to facilitate better access under skin flaps, or may be configured to be flexible to facilitate imaging surgical cavities with complex geometries.

Distal end 4116 includes one or more light sources, such as light-emitting diodes (LEDs) configured to emit light having a specific wavelength, as discussed above. Distal end 4116 may also include a camera sensor having a lens and one or more optical filters, as discussed above. In imaging device 4000, the excitation light source may be configured to cause porphyrins in tumor cells to fluoresce. When illuminated by 405 nm excitation light, the porphyrins and, thus, tumor cells fluoresce red. The porphyrins in the tumor cells and in other cancerous tissue may be induced by administration of a compound such as 5-ALA to a patient prior to surgery. In addition to using the imaging device 4000 to examine the surgical cavity, the device can be used with tissue specimens excised during surgery. The imaging drape can be attached to the imaging device to position the excised tissue in the darkened portable imaging environment while the specimen is subject to FL imaging. The imaging device may have other excitation light sources of different wavelengths which may also be used with an imaging drape in accordance with the present disclosure,

The imaging device and drape, according to the various embodiments disclosed herein, may be used with a sterile shield 5000 to provide a barrier between a nonsterile imaging device contained in the shield and the sterile field of surgery, for example, the sterile field within a surgical operation room. Such may allow the non-sterile device, fully contained in shield 5000, to be used in a sterile environment. Thus, shield 5000 may be a surgical drape that contains the nonsterile environment from exposing the sterile environment,

FIG. 15B shows shield 5000 disposed over imaging device 4000, and FIG. 15A shows a cross-sectional view of a distal end portion of the imaging device with the shield disposed over it. Although FIGS. 15A and 15B show shield 5000 as being used with imaging device 4000, it is contemplated that shield 5000 may be used with any of the imaging devices disclosed herein, as well as other devices, and shaped to accommodate whatever device it is used with.

Shield 5000, when disposed on the imaging device, may extend from a proximal end to a distal end of the device, for example, from first end portion 4112 to second end portion 4114 of the imaging device. A proximal portion of shield 5000 may include a closing mechanism to secure shield 5000 on the imaging device. Shield 5000 may comprise a polymer material, such as polyethylene, polyurethane, or other polymer materials. In some embodiments, shield 5000 is transparent so that a user may view the imaging device through shield 5000.

As shown in FIG. 15B, shield 5000 may be shaped to mate with and fit the contours of the imaging device. Shield 5000 may be coupled and disposed over the imaging device prior to attachment of the imaging device to the imaging drape.

FIG. 15A shows a distal end portion of shield 5000 including a distal end cap 5010, which is connected to the distal end of the polymer material of shield 5000. Distal end cap 5010 may be a lens formed of a plastic material such as, for example, acrylic or polycarbonate so as not to affect the optics of imaging device 4000. In some embodiments, distal end cap 5010 is welded to the polymer material of shield 5000. Additionally, distal end cap 5010 may be transparent, similar to the polymer material of shield 5000. Thus, the FL and/or white-light emitted from the imaging device may penetrate through the transparent lens of distal end cap 5010 and into the interior environment of the drape.

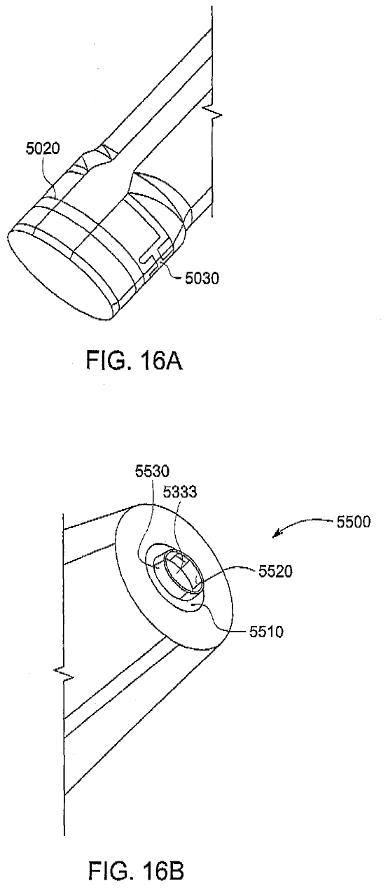

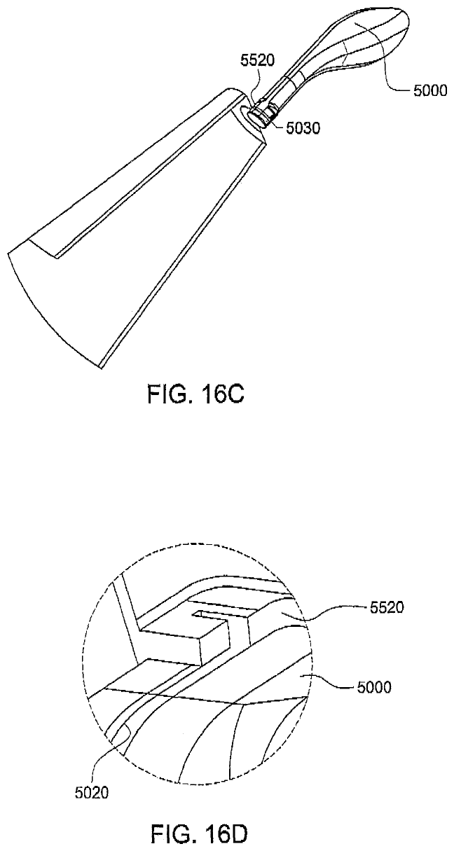

FIG. 15C shows an exemplary embodiment in which shield 5000 is disposed over the imaging device and the device is coupled to the drape for imaging a patient using FL and/or white-light imaging. The drape in FIG. 15C may be any of the drape embodiments disclosed herein. As shown in FIGS. 15A-18D, the drape and shield may each have mating retaining features to couple the imaging device to the drape. In some embodiments, distal end cap 5010 includes a connecting element 5020 that is configured to mate with a support ring on a connecting element on the drape, as discussed further below. The mating connection between connecting element 5020 and the support ring may provide a secure attachment between the imaging device and the drape, and may reduce/block all light from entering the interior of the drape. As shown in FIGS. 15A and 16A, connecting element 5020 may be an indentation in distal end cap 5010. For example, connecting element 5020 may be a circumferential groove in distal end cap 5010. In some embodiments, connecting element 5020 may be a breakable member that is configured to break first when the imaging device is disconnected from the drape. As discussed further below, the breakable feature of connecting element 5020 may prevent the shield from being reused.

Distal end cap 5000 may also include an indicating feature 5030 used to help guide the connection of shield 5000 with a drape. As shown in FIG. 15A, indicating feature 5030 may be an indentation in a distal end cap 5010. Indicating feature 5030 may be used to properly align the drape with the imaging device. Thus, indicating feature 5030 may be used as an indication to a user to not place the drape material proximal of indicating feature 5030. For example, indicating feature 5030 may be a circumferential groove in distal end cap 5010.

The retaining feature on the drape may be connecting element 5500, as shown in FIG. 16B. Connecting element 5500 may include a mount 5510 that is attached to the drape body. Mount 5510 may be welded to the drape, or attached via adhesives, tape, Velcro, staples, etc, A support ring 5520 may extend outward from the mount 5510. One or more sidewalls 5530 may brace support ring 5520 on mount 5510. An opening 5333 may be defined in mount 5510 such that opening 5333 is bordered by sidewalls 5530. Opening 5333 may be aligned with opening 133 on the drape, as discussed above. At least a portion of distal end cap 5010 may be disposed in opening 5333 when the imaging device is secured to the drape.

Connecting element 5020 on shield 5000 may be configured to mate with support ring 5520 on connecting element 5500 in order to secure the imaging device to the drape. Thus, support ring 5520 may be configured to be nested within the circumferential groove of connecting element 5020 to securely fasten the imaging device to the drape. In some embodiments, connecting element 5020 and support ring 5520 form a male-female locking mechanism. It is also contemplated that these components may be coupled together via an interference fit.

FIG. 16C shows connecting element 5020 and support ring 5520 interacting to secure the imaging device to the drape. Such may allow the imaging device to obtain a FL and/or white-light image within the darkened interior environment of the drape, as discussed above. Removal and disconnection of the imaging device from the drape causes the shield to break at connecting element 5020. For example, disconnecting the imaging device from the drape causes the connecting element 5020 to bend and flex on support ring 5520. This bending and flexing causes the connecting element 5020 to break. Thus, distal end cap 5010 is separated from the remainder of the shield at connecting element 5020. Because connecting element 5020 is a groove/indentation in shield, it is configured to break first when the imaging device is removed from the drape. However, it is also contemplated that connecting element 5020 may be a weakened or thinner piece of material on the shield to provide the breakable nature of this component. The separation of distal end cap 5020 from the remainder of the shield prevents the shield from being reused.

In the embodiment of FIG. 16B, connecting element 5500 does not include projections to prevent the material of the drape from collapsing into the field of view. In this embodiment, connecting element 5500 may be used with a relatively smaller drape that does have excess material that may inadvertently move into the field of view.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

Further, this description's terminology is not intended to limit the disclosure. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly. Likewise, descriptions of movement along and around various axes includes various special device positions and orientations. In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition,

Those of ordinary skill in the art will understand that embodiments of the present disclosure contemplate various mechanisms, which include various configurations of features, for the material of the drape, shaping elements for the drape, connecting elements for connecting the drape to a portable handheld imaging device. As will be apparent after reading the disclosure, other materials and/or structures may be used for these mechanisms without departing from the scope of the application.

Furthermore, the devices and methods may include additional components or steps that were omitted from the drawings for clarity of illustration and/or operation. Accordingly, this description is to be construed as illustrative only and is for the purpose of teaching those skilled in the art the general manner of carrying out the present disclosure. It is to be understood that the various embodiments shown and described herein are to be taken as exemplary. Elements and materials, and arrangements of those elements and materials, may be substituted for those illustrated and described herein, parts and processes may be reversed, and certain features of the present disclosure may be utilized independently, all as would be apparent to one skilled in the art after having the benefit of the description herein. Changes may be made in the elements described herein without departing from the spirit and scope of the present disclosure and following claims, including their equivalents.

It is to be understood that the particular examples and embodiments set forth herein are non-limiting, and modifications to structure, dimensions, materials, and methodologies may be made without departing from the scope of the present disclosure.

It should be understood that while the present disclosure has been described in detail with respect to various exemplary embodiments thereof, it should not be considered limited to such, as numerous modifications are possible without departing from the broad scope of the appended claims, including the equivalents they encompass.

1. A drape, comprising:
a drape body, wherein the drape body is configured to limit passage of electromagnetic radiation through the drape body to an interior imaging environment defined by the drape body such that electromagnetic radiation within the interior imaging environment does not exceed a predetermined threshold; and
a connecting element permanently coupled to the drape body and defining a hole in the drape body, the connecting element being configured to attach the drape to an imaging device.

2. The drape of claim 1, wherein an amount of electromagnetic radiation above the predetermined threshold is an amount of electromagnetic radiation known to interfere with fluorescence and white light imaging.

3. The drape of claim 1, wherein the connecting element further comprises at least one support structure positioned on an interior or an exterior surface of the drape.

4. (canceled)

5. The drape of claim 3, wherein the at least one support structure is configured to support a portion of the drape body, surrounding the connecting element, when the drape is in a deployed configuration such that the portion of the drape extends in a planar fashion from the connecting element to a first shaping element of the drape.

6. The drape of claim 3, wherein the at least one support structure comprises a plurality of support structures extending radially outward or circumferentially from a frame of the connecting element.

7. (canceled)

8. The drape of claim 3, wherein the at least one support structure is a finger element.

9. The drape of claim 8, wherein the at least one support structure includes a plurality of finger elements extending from a frame of the connecting element.

10. (canceled)

11. The drape of claim 5, wherein the portion of the drape body extending to the first shaping element is substantially circular.

12. The drape of claim 5, wherein the first shaping element is a flexible structure embedded in the drape body.

13. The drape of claim 5, wherein the first shaping element is configured to maintain a shape of the drape body in the deployed configuration.

14. The drape of claim 5, wherein the first shaping element is configured to cooperate with the at least one support structure to hold the drape body out of a field of view of the imaging device.

15. The drape of claim 5, wherein the first shaping element is further configured to change a shape of the drape in response to user manipulation and to maintain the change of shape after release by a user.

16. The drape of claim 1, further comprising at least one valve configured to permit air flow into the drape during deployment of the drape, the at least one valve being a one-way valve configured to prevent air from escaping from the interior imaging environment after deployment of the drape.

17. (canceled)

18. The drape of claim 1, wherein the drape has a substantially circular shape in an undeployed configuration.

19. The drape of claim 18, wherein the drape has a substantially cylindrical shape or a truncated cone shape in a deployed configuration.

20. (canceled)

21. The drape of claim 5, further comprising a second shaping element, the second shaping element having a substantially circular shape and being positioned radially outward from the first shaping element when the drape is in an undeployed configuration.

22. (canceled)

23. (canceled)

24. The drape of claim 1, wherein the connecting element comprises a frame, and wherein the hole in the drape body is formed in the frame of the connecting element.

25. (canceled)

26. The drape of claim 24, wherein the frame is substantially rectangular in shape.

27. The drape of claim 1, wherein the hole in the drape body is shaped and sized to receive a portion of the imaging device containing a light source and a lens.

28. The drape of claim 27, wherein the hole is substantially kidney shaped.

29. The drape of claim 1, wherein the connecting element further comprises clips configured to attach to the imaging device, the clips being movable between an open position and a closed position.

30. (canceled)

31. The drape of claim 29, further comprising a cover portion configured to sit between the hole in the drape body and the clips, wherein the clips are configured to move to the open position during removal of the cover portion.

32. (canceled)

33. The drape of claim 1, wherein, when in a deployed configuration, the drape includes a top portion that extends substantially radially around the connecting element and a skirt portion that extends vertically from a perimeter of the top portion of the drape.

34. The drape of claim 33, wherein the skirt portion is flexible.

35. The drape of claim 33, wherein the skirt portion is substantially rigid.

36. The drape of claim 33, wherein a bottom of the skirt portion includes one or more fixing elements configured to fix the bottom of the skirt portion of the drape relative to an area to be imaged, such that the area to be imaged is positioned or contained within the interior imaging environment of the drape.

37. The drape of claim 36, wherein the one or more fixing elements include at least one of a weighted element, a magnet, a draw string, tape, a wire, and a hook and loop closure.

38. The drape of claim 1, wherein the drape comprises at least one of a plastic material, a paper material, a vinyl material, and/or a cloth material.

39. The drape of claim 38, wherein the drape further comprises a coating configured to limit passage of electromagnetic radiation, and wherein an interior surface of the drape, which defines the interior imaging environment, is coated with a material to reduce reflection within the interior imaging environment.

40. (canceled)

41. A system for fluorescence-based imaging of a target, comprising:
the drape of claim 1; and
a portable, handheld imaging device, wherein the imaging device includes a portion with a lens and at least one blue/violet light source, and wherein the connecting element is configured to encompass an area surrounding the lens and the at least one light source to position the light source within the interior imaging environment of the drape body.

42. The system of claim 41, further comprising a sterile shield configured to be disposed over a portion of the imaging device.

43-128. (canceled)

129. The drape of claim 1, wherein the drape is configured for single-use and is disposable.

130. (canceled)

131. A drape, comprising:
a drape body, wherein the drape body is configured to limit passage of ambient light through the drape body to an interior imaging environment defined by the drape body; and
a connecting element coupled to the drape body and defining a hole in the drape body, the connecting element including a planar surface and fingers that project outwardly from the planar surface, and the connecting element being configured to attach the drape to an imaging device.

132-196. (canceled)

197. The drape of claim 1, wherein the drape has a truncated pyramid shape in a deployed configuration.
